(19) United States
(12) Patent Application Publication
(10) Pub. No.: US 2026/0283676 A1
(43) Pub. Date: Sep. 24, 2026

(54) ELECTROSURGICAL CONNECTION UNIT

(71) Applicant: CMR SURGICAL LIMITED, Cambridge, Cambridgeshire (GB)

(72) Inventor: Benjamin Miles Huggett, Hertfordshire (GB)

(21) Appl. No.: 19/477,306

(22) PCT Filed: Apr. 26, 2024
(86) PCT No.: PCT/GB2024/051094

§ 371 Date: Oct. 21, 2025

(30) Foreign Application Priority Data
Apr. 28, 2023 (GB) 2306379.5

(51) Int. Cl. A61B 18/12; A61B 18/00; A61B 34/30

(52) U.S. Cl. CPC A61B 18/1206; A61B 34/30; A61B 2018/00178; A61B 2018/00708; A61B 2018/00755; A61B 2018/0094

(57) ABSTRACT

Electrosurgical connection units for a surgical robot arm. The electrosurgical connection units include: an input port connectable to an electrosurgical generator; an output port connectable to an electrosurgical instrument attached to the surgical robot arm; one or more activation switch units, wherein activation of an activation switch unit of the one or more activation switch units causes an activation signal to be output from the input port, the activation signal is configured to, when the input port is connected to the electrosurgical generator, cause the electrosurgical generator to generate a driving electrosurgical signal which is received on the input port; a connection switch unit, wherein activation of the connection switch unit causes a portion of the input port to be electrically connected to the output port such that, when the input port is connected to the electrosurgical generator, the driving electrosurgical signal received on the input port is output on the output port; and a control unit configured to, in response to receiving a control signal from an external computing device, cause the connection switch unit to be activated and subsequently cause an activation switch unit of the one or more activation switch units to be activated.

### BACKGROUND

It is known to use robots for assisting and performing surgery. FIG. 1 illustrates a typical surgical robot 100 which comprises a base 108, an arm 102, and an instrument 105. The base supports the robot, and is itself attached rigidly to, for example, the operating theatre floor, the operating theatre ceiling, or a trolley. The arm extends between the base and the instrument. The arm is articulated by means of multiple flexible joints 103 along its length, which are used to locate the surgical instrument in a desired location relative to the patient. The surgical instrument is attached to the distal end 104 of the robot arm. The surgical instrument penetrates the body of the patient 101 at a port 107 so as to access the surgical site. At its distal end, the instrument comprises an end effector 106 for engaging in a medical procedure.

A variety of surgical instruments are known, each adapted to perform a particular surgical function. FIG. 2 illustrates an example surgical instrument 200. The surgical instrument comprises a base 201 by means of which the surgical instrument connects to the robot arm. A shaft 202 extends between the base 201 and an articulation 203. The articulation 203 terminates in an end effector 204. The articulation 203 permits the end effector 204 to move relative to the shaft 202. It is desirable for at least two degrees of freedom to be provided to the motion of the end effector 204 by means of the articulation.

An electrosurgical instrument is a surgical instrument adapted to perform electrosurgery. As is known to those of skill in the art, electrosurgery is the passing of a high frequency (i.e. radio frequency) current through tissue to cause a desired effect (e.g. cutting the tissue or coagulating the tissue). Although the term electrosurgery is often used interchangeably with the term electrocautery, electrosurgery and electrocautery are separate and distinct procedures. Where electrocautery uses heat conduction from a probe heated by a direct current (DC), electrosurgery uses radio frequency (RF) alternating current (AC) to heat the tissue by RF induced intracellular oscillation of ionized molecules that result in an intracellular temperature. Accordingly, during electrosurgery the patient is included in the circuit and current enters the patient's body, whereas during electrocautery current does not enter the patient's body.

There are two types of electrosurgery-monopolar and bipolar. In monopolar electrosurgery the high frequency current passes through the patient from a live or active electrode of the electrosurgical instrument to a separate return electrode placed on the patient, which may also be referred to as a dispersive electrode pad, a grounding pad, a neutral electrode, a grounding mat, an indifferent electrode, or a patient electrode. In bipolar electrosurgery the active and return electrodes are both within the electrosurgical instrument and the current passes through the patient from the active electrode of the electrosurgical instrument to the return electrode of the electrosurgical instrument. An electrosurgical instrument which is configured for monopolar electrosurgery (e.g. an electrosurgical instrument that comprises an active electrode only) will be referred to herein as a monopolar electrosurgical instrument, and an electrosurgical instrument which is configured for bipolar electrosurgery (e.g. an electrosurgical instrument that comprises both an active electrode and a return electrode) will be referred to herein as a bipolar electrosurgical instrument.

Electrosurgical instruments receive the high frequency current (which is referred to herein as a driving electrosurgical signal) from an electrosurgical generator, which may also be referred to as an electrosurgery generator, electrosurgical end unit, electrosurgery end unit, or ESU. Electrosurgical generators are generally capable of generating multiple different current waveforms to achieve different surgical effects. For example, many standard electrosurgical generators can be configured to generate COAG, CUT and BLEND waveforms. The COAG waveform consists of bursts of radio frequency, which, when used at a low power setting, causes a desiccation effect, and, when used at a high-power setting, causes a fulguration effect. The CUT waveform is a continuous waveform at lower voltage, but higher current than COAG, which causes the tissue to be cut. A BLEND waveform is essentially a CUT waveform with a lower duty cycle than a CUT waveform. A BLEND waveform typically has a duty cycle between 15% to 75% whereas a CUT waveform typically has a duty cycle greater than 75%. The off time allows the tissue to cool creating some haemostasis. Accordingly, a BLEND waveform is used where haemostasis is required as tissue is cut. It will be evident to a person of skill in the art that these are examples only and that different electrosurgical generators may be configured to generate different and/or additional waveforms.

In existing manual, as opposed to robotic, electrosurgical systems, the surgeon can cause a driving electrosurgical signal with a particular waveform (e.g. COAG, CUT or BLEND) to be provided to an electrosurgical instrument attached to the electrosurgical generator using controls (e.g. buttons) on the manual electrosurgical instrument or using controls (e.g. foot pedals) connected to the electrosurgical generator. FIG. 3 illustrates an example manual monopolar electrosurgical instrument 302 that comprises at its distal end 304 an active electrode 306 for achieving a surgical effect when activated by a driving electrosurgical signal. The manual monopolar electrosurgical instrument 302 also comprises two activation buttons 308, 310 which can be used to cause the electrosurgical generator to provide a driving electrosurgical signal with a first waveform (e.g. a CUT waveform) and a driving electrosurgical signal with a second waveform (e.g. a COAG waveform) respectively to the electrosurgical instrument 302. The CUT button is typically coloured yellow and the COAG button is typically coloured blue to comply with specific standards.

FIG. 4 is used to explain how such a monopolar electrosurgical instrument 302 controls the operation of an electrosurgical generator 402. As shown in FIG. 4, the electrosurgical instrument 302 is attached to the electrosurgical generator 402 via a cable 404. Typically, the cable is integrated with the electrosurgical instrument 302 to form a single disposable device. However, the cable 404 may not be integral with the electrosurgical instrument, but the cable 404 may have a connector at one end which is configured to engage a corresponding connector of the electrosurgical instrument 302. In either case, the cable 404 typically comprises a connector at one end which is configured to engage a corresponding connector of the electrosurgical generator 402. This generator-end connector of the cable 404 may, for example. Be a standard 3-pin Valleylab™ connector, such as that described in the Valleylab™ FT10 Energy Platform User Guide.

The cable 404 carries three conductors or wires 410, 412, 414—an active wire 410 and two control wires 412, 414. The active wire 410 is used to transmit a driving electrosurgical signal generated by the electrosurgical generator 402 to the electrosurgical instrument 302. The control wires 412, 414 are used to transmit activation signals generated by the electrosurgical instrument 302 to the electrosurgical generator 402.

The active wire 410 is electrically coupled to the active electrode 306 of the electrosurgical instrument 302 so that any driving electrosurgical signal received on the active wire 410 is provided to the active electrode 306. The two activation buttons 308, 310 of the electrosurgical instrument 302 are connected to corresponding switches 406, 408. One port of each switch 406, 408 is coupled to the active wire 410 and the other port of each switch 406, 408 is coupled to one of the control wires 412, 414. Specifically, a second port of the first switch 406 is coupled to the first control wire 412, and the second port of the second switch 408 is coupled to the second control wire 414. When an activation button 308, 310 is depressed the corresponding switch 406, 408 is activated which connects the active wire 410 to the corresponding control wire 412, 414 which sends a corresponding activation signal to the electrosurgical generator 402. Specifically, when the electrosurgical generator 402 is powered on, but is not active (i.e. is not generating a driving electrosurgical signal) the electrosurgical generator 402 outputs a weak signal on the active wire 410 and when a switch 406, 408 is activated that weak signal is transmitted on the corresponding control wire 412, 414.

When an activation signal is detected by control logic 416 of the electrosurgical generator the control logic 416 causes RF generation logic 418 of the electrosurgical generator 402 to output a driving electrosurgical signal on the active wire 410 with a waveform associated with that activation signal. For example, the first activation button 308 may be associated with a CUT waveform such that, when the user presses or activates the first activation button 308, a first activation signal is transmitted to the electrosurgical generator 402 on the first control wire 412 which causes the electrosurgical generator 402 to output a driving electrosurgical signal with a CUT waveform on the active wire 410. The second activation button 310 may be associated with a COAG waveform such that, when the user presses or activates the second activation button 310, a second activation signal is transmitted to the electrosurgical generator 402 on the second control wire 414 which causes the electrosurgical generator 402 to output a driving electrosurgical signal with a COAG waveform on the active wire 410. In this example, a separate return electrode 420 is directly connected to the electrosurgical generator 402 via a separate cable 422.

Instead of having the activation buttons on the electrosurgical instrument itself there may be a foot pedal system which allows the surgeon, or other user, to cause an electrosurgical generator to provide a driving electrosurgical signal with one waveform or another to an electrosurgical instrument attached to the electrosurgical generator. In some cases, using a separate foot pedal system is preferred as it reduces the complexity of the electrosurgical instrument. FIG. 5 illustrates an example foot pedal system 502 comprising a first foot pedal 504 and a second foot pedal 506 which can be used to cause an electrosurgical generator to provide a driving electrosurgical signal with a first waveform (e.g. a CUT waveform) and a second waveform (e.g. a COAG waveform) respectively to an electrosurgical instrument attached to the electrosurgical generator. FIG. 6 is used to explain how such a foot pedal system 502 can be used to control the operation of an electrosurgical generator 402 and an electrosurgical instrument 602.

The foot pedal system 502 is connected to the electrosurgical generator 402 via a cable 604. The cable 604 may be integral with the foot pedal system 502 or may be connectable to the foot pedal system 502 via a connector which engages a corresponding connector of the foot pedal system. In either case, the cable 604 typically comprises a connector that engages a corresponding connector of the electrosurgical generator 402. In these cases the cable 604 carries three conductors or wires 610, 612, 614—an active wire 610 and two control wires 612, 614.

The foot pedals 504 and 506 work in the same manner as the activation buttons 308, 310 of FIGS. 3-4. Specifically, the foot pedals 504, 506, like the activation buttons 308, 310, are each connected to a switch 606, 608. One port of each switch 606, 608 is coupled to the active wire 610 and a second port of each switch 606, 608 is coupled to one of the control wires 612, 614. In particular, the second port of the first switch 606 is coupled to the first control wire 612 and the second port of the second switch 608 is coupled to the second control wire 614. When a foot pedal 504, 506 is depressed the corresponding switch 606, 608 is activated which connects the active wire 610 to the corresponding control wire 612, 614 which causes a corresponding activation signal to be transmitted to the electrosurgical generator 402.

When an activation signal is detected by the control logic 416 of the electrosurgical generator 402, the control logic 326 causes the RF generation logic 418 of the electrosurgical generator 402 to output a driving electrosurgical signal with a waveform associated with that activation signal on the active wire 610. For example, the first foot pedal 504 may be associated with a CUT waveform such that when the user presses or activates the first foot pedal 504 a first activation signal is transmitted to the electrosurgical generator 402 on the first control wire 612. When the electrosurgical generator 402 detects the first activation signal, the electrosurgical generator 402 outputs a driving electrosurgical signal with a CUT waveform on the active wire 610. The second foot pedal 506 may be associated with a COAG waveform such that when the user presses or activates the second foot pedal 506 a second activation signal is transmitted to the electrosurgical generator 402 on the second control wire 614. When the electrosurgical generator 402 detects the second activation signal, the electrosurgical generator 402 outputs a driving electrosurgical signal with a COAG waveform on the active wire 610. In this example, a cable comprising a single wire used to carry the driving electrosurgical signal is then connected to the active electrode 616 of the electrosurgical instrument 602. Like the example in FIG. 4, a separate return electrode 420 is directly connected to the electrosurgical generator 402 via a separate cable 422.

While the activation buttons 308, 310 and foot pedals 504, 506 described above with respect to FIGS. 3-6 provide convenient and safe means for activating an electrosurgical instrument in a manual electrosurgical system where a surgeon, or other user, holds the electrosurgical instrument during surgery, they are typically not suitable or not convenient for activating an electrosurgical instrument in a robotic electrosurgical system. Specifically, robotic electrosurgical systems typically comprise a plurality of robotic arms, each of which can be attached to a different surgical instrument. A surgeon, or other user, can dynamically control any of the arms (and thus any of the surgical instruments attached thereto) via one or more input controllers (e.g. hand controllers) of a central command interface (which may also be referred to as a surgeon console).

If an electrosurgical instrument in a robotic electrosurgical system comprised one or more activation buttons, as described with respect to FIGS. 3-4, the surgeon would either have to move away from the command interface to depress the appropriate button on the instrument, or, the surgeon, or other user, would have to instruct another person to do so, which may be unsafe (e.g. depressing the button may cause the instrument to move to an undesired position in the patient) and may cause unnecessary delays and errors. If, alternatively, a foot pedal system is connected to each electrosurgical generator, since there may be multiple generators that control different electrosurgical instruments the surgeon, or other user, may have to manually confirm that the correct pedal system is used. Foot pedals can also prevent, or at least make it difficult for, the surgeon, or other user, to operate the electrosurgical instrument whilst standing up, which may be desirable in some circumstances. It would be much more convenient and safer if the surgeon, or other user, were able to active an electrosurgical instrument attached to a robot arm via the command interface. Specifically, not only would it be more convenient to activate electrosurgical instruments from the command interface, but the system could ensure that the desired electrosurgical instrument was being activated.

The embodiments described below are provided by way of example only and are not limiting of implementations which solve any or all of the disadvantages of electrosurgical systems.

### SUMMARY

This summary is provided to introduce a selection of concepts that are further described below in the detailed description. This summary is not intended to identify key features or essential features of the claimed subject matter, nor is it intended to be used to limit the scope of the claimed subject matter.

Described herein are electrosurgical connection units for a surgical robot arm. The electrosurgical connection units include: an input port connectable to an electrosurgical generator; an output port connectable to an electrosurgical instrument attached to the surgical robot arm; one or more activation switch units, wherein activation of an activation switch unit of the one or more activation switch units causes an activation signal to be output from the input port, the activation signal is configured to, when the input port is connected to the electrosurgical generator, cause the electrosurgical generator to generate a driving electrosurgical signal which is received on the input port; a connection switch unit, wherein activation of the connection switch unit causes a portion of the input port to be electrically connected to the output port such that, when the input port is connected to the electrosurgical generator, the driving electrosurgical signal received on the input port is output on the output port; and a control unit configured to, in response to receiving a control signal from an external computing device, cause the connection switch unit to be activated and subsequently cause an activation switch unit of the one or more activation switch units to be activated.

A first aspect provides an electrosurgical connection unit for a surgical robot arm, the electrosurgical connection unit comprising: an input port connectable to an electrosurgical generator; an output port connectable to an electrosurgical instrument attached to the surgical robot arm; one or more activation switch units, wherein activation of an activation switch unit of the one or more activation switch units causes an activation signal to be output from the input port, the activation signal is configured to, when the input port is connected to the electrosurgical generator, cause the electrosurgical generator to generate a driving electrosurgical signal which is received on the input port; a connection switch unit, wherein activation of the connection switch unit causes a portion of the input port to be electrically connected to the output port such that, when the input port is connected to the electrosurgical generator, the driving electrosurgical signal received on the input port is output on the output port; and a control unit configured to, in response to receiving a control signal from an external computing device, cause the connection switch unit to be activated and subsequently cause an activation switch unit of the one or more activation switch units to be activated.

The control unit may be configured to cause the connection switch unit to be activated and subsequently cause the activation switch unit of the one or more activation switch units to be activated by outputting a first switch control signal to control the connection switch unit and a second switch control signal to control the activation switch unit.

The first and second switch control signals may be the same.

The electrosurgical connection unit may further comprise a first circuit that is situated between the control unit and the connection switch unit and is configured to convert the first switch control signal to a signal to activate the connection switch unit, and a second circuit situated between the control unit and the activation switch unit and configured to convert the second switch control signal to a signal to activate the activation switch unit, wherein the first and second circuits are configured to, when the first and second control signals are the same, activate the connection switch unit prior to activating the activation switch unit.

The first and second switch control signals may be oscillating signals and the first and second circuits may be first and second conversions circuits which are configured to convert an oscillating signal to a control signal.

The first conversion circuit may be configured differently from the second conversion circuit such that when the first and second switch control signals are the same, the connection switch unit is activated prior to the activation switch unit.

Each of the first and second conversion circuits may comprise a low pass filter and a timing constant of the low pass filter of the first conversion circuit may differ from a timing constant of the low pass filter of the second conversion circuit.

The low pass filter of each of the first and second conversion circuits may comprise a capacitor and one or more resistors, and a resistance value of at least one of the one or more resistors of the low pass filter of the first conversion circuit may differ from a resistance value of a corresponding resistor of the low pass filter of the second conversion circuit.

The control unit may be configured to, in response to ceasing to receive the control signal, deactivate the activation switch unit and subsequently deactivate the connection switch unit.

The control unit may be configured to deactivate the activation switch unit and subsequently deactivate the connection switch unit by ceasing to output the first and second switch control signals.

The first and second circuits may be configured to, in response to ceasing to receive the first and second control signals at a same time, cause the connection switch unit to be deactivated subsequent to causing the activation switch unit to be deactivated.

The input port may be coupled to an input active wire for receiving the driving electrosurgical signal from the input port. The output port may be coupled to an output active wire for providing the driving electrosurgical signal to the output port. The connection switch unit may, when activated, electrically connects the input active wire and the output active wire.

When the output port is connected to the electrosurgical instrument, outputting the driving electrosurgical signal on the output port may cause a return electrosurgical signal to be received on the output port, and the electrosurgical connection unit may further comprise a second connection switch unit, and activation of the second connection switch unit causes another portion of the input port to be electrically connected to the output port such that the return electrosurgical signal received on the output port is output on the input port.

The input port may be coupled to (i) an input active wire for receiving the driving electrosurgical signal from the input port and (ii) one or more control wires for transmitting an activation signal to the input port, and when an activation switch unit is activated the input active wire may be electrically connected to one of the one or more control wires to generate the activation signal.

Each activation switch unit of the one or more activation switch units may comprise one or more switches in series and one end of the one or more switches may be coupled to the input active wire and the other end of the one or more switches may be coupled to one of the one or more control wires.

The one or more activation switch units may comprise a first activation switch unit and a second activation switch unit.

When the input port is connected to the electrosurgical generator, activating the first activation switch unit may cause a first activation signal to be transmitted to the electrosurgical generator which causes the electrosurgical generator to output a driving electrosurgical signal with a first waveform, and activating the second activation switch unit may cause a second activation signal to be transmitted to the electrosurgical generator which causes the electrosurgical generator to output a driving electrosurgical signal with a second waveform.

The electrosurgical connection unit may further comprise an isolation device that forms an isolation barrier between (i) the one or more activation switch units and the connection switch unit and (ii) the control unit.

The electrosurgical connection unit may further comprise an alternating current coupling circuit for each activation switch unit of the one or more activation switch units, each alternating current coupling circuit being situated between the control unit and the corresponding activation switch unit of the one or more activation switch units, each alternating current coupling circuit configured to receive an alternating current switch control signal output by the control unit and generate a direct current filtered version of the alternating current switch control signal.

The electrosurgical connection unit may further comprise a measurement unit configured to measure a parameter of an activation switch unit of the one or more activation switch units or the connection switch unit and output measurement information to the control unit, the measurement information enabling a determination to be made as whether the activation switch unit or the connection switch unit is operating as expected.

The electrosurgical connection unit may further comprise a capacitance emulation unit configured to, when the electrosurgical generator is connected to the input port, add or subtract capacitance to an electrical path between the electrosurgical connection unit and the electrosurgical generator.

The control unit may be configured to periodically generate a token comprising information indicating a time at which the token was generated, and transmit the token to an external computing device, and the control signal is a modified version of one of the tokens.

The control unit may be configured to only activate the connection switch unit and the activation switch unit of the one or more activation switch units in response to receiving the modified version of the token when, at the time the modified version of the token is received at the control unit, an elapsed time since the token was generated is less than a threshold.

A second aspect provides a surgical robot arm comprising the electrosurgical connection unit of the first aspect.

A second aspect provides a surgical robotic system comprising: the surgical robot arm of the second aspect; the electrosurgical generator connected to the input port of the electrosurgical connection unit; and the electrosurgical instrument connected to the output port of the electrosurgical connection unit.

### BRIEF DESCRIPTION OF THE DRAWINGS

Examples will now be described in detail with reference to the accompanying drawings in which:

FIG. 1 is a schematic diagram of an example surgical robot performing a surgical procedure;

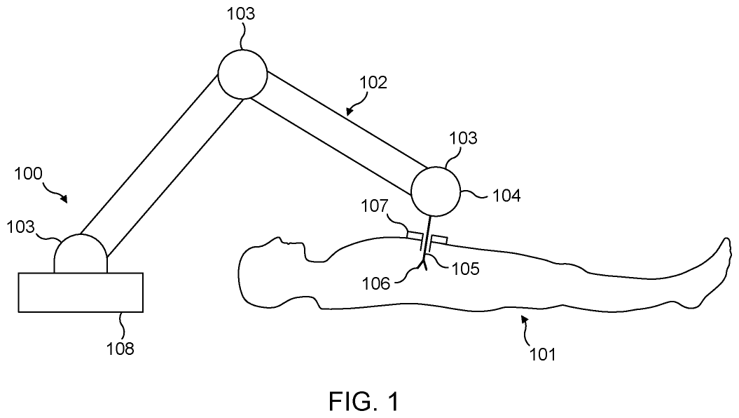

FIG. 2 is a schematic diagram of an example surgical instrument;

FIG. 3 is a schematic diagram of an example monopolar electrosurgical instrument with two activation buttons;

FIG. 4 is a block diagram of an example electrosurgical system comprising the monopolar electrosurgical instrument of FIG. 3, an electrosurgical generator and a return electrode;

FIG. 5 is a schematic diagram of an example foot pedal system that can be used to control an electrosurgical generator;

FIG. 6 is a block diagram of an example electrosurgical system comprising the foot pedal system of FIG. 5, a monopolar electrosurgical instrument, an electrosurgical generator and a return electrode;

FIG. 7 is a schematic diagram of an example surgical robot system including a robot arm comprising an electrosurgical connection unit;

FIG. 8 is a bock diagram of an example electrosurgical system comprising a first example electrosurgical connection unit for a monopolar electrosurgical instrument;

FIG. 9 is a block diagram of a second example electrosurgical connection unit for a monopolar electrosurgical instrument comprising a plurality of conversion circuits;

FIG. 10 is a circuit diagram of example implementations for the conversion circuits of FIG. 9;

FIG. 11 is a signal graph illustrating the effect of an input signal on the conversion circuits of FIG. 10;

FIG. 12 is a block diagram of an example electrosurgical system comprising a third example electrosurgical connection unit for a monopolar electrosurgical instrument;

FIG. 13 is a block diagram of an example electrosurgical system comprising an example electrosurgical connection unit for a bipolar electrosurgical instrument; and

FIG. 14 is a block diagram of an example method which may be implemented by the control unit of any of the electrosurgical connection units of FIGS. 8, 9, 12 and 13 for causing a driving electrosurgical signal to be output on the output port of the electrosurgical connection unit.

The accompanying drawings illustrate various examples. The skilled person will appreciate that the illustrated element boundaries (e.g., boxes, groups of boxes, or other shapes) in the drawings represent one example of the boundaries. It may be that in some examples, one element may be designed as multiple elements or that multiple elements may be designed as one element. Common reference numerals are used throughout the figures, where appropriate, to indicate similar features.

### DETAILED DESCRIPTION

The following description is presented by way of example to enable a person skilled in the art to make and use the invention. The present invention is not limited to the embodiments described herein and various modifications to the disclosed embodiments will be apparent to those skilled in the art. Embodiments are described by way of example only.

As described above, it is desirable from each of a safety, efficiency, and convenience perspective, to be able to activate an electrosurgical instrument attached to a surgical robot arm from the command interface (e.g. surgeon console). Accordingly, the Applicant developed an electrosurgical connection unit for a surgical robot arm that is connectable to an electrosurgical generator, via an input port thereof, and to an electrosurgical instrument attached to the surgical robot arm, via an output port thereof. The electrosurgical connection unit is configured to, in response to receiving one or more control signals from an external computing device (such as, but not limited, to the command interface), output an activation signal on the input port which causes an electrosurgical generator connected thereto to provide a driving electrosurgical signal to the input port. The input port of the electrosurgical connection unit is coupled to the output port thus a driving electrosurgical signal received on the input port is output on the output port, and thus to an electrosurgical instrument connected thereto More particularly, the electrosurgical connection unit comprises a control unit and one or more activation switch units and when the control unit receives a control signal indicating that an electrosurgical instrument attached thereto is to be activated or energised, the control unit is configured to activate one of the activation switch units which causes an activation signal to be output on the input port. The activation signal, when received at an electrosurgical generator, causes the electrosurgical generator to output a driving electrosurgical signal which is received at the input port of the electrosurgical connection unit and output on the output port thereof. This electrosurgical connection unit is described in the Applicant's GB patents 2571566 and 2572688.

In some cases, multiple electrosurgical instruments, each attached to a different robot arm, may be used in a procedure (e.g. surgery). Therefore, in such cases, there may be multiple electrosurgical instruments present in a surgical field simultaneously. This can create a capacitive coupling problem since it increases the number of available current paths. Specifically, as described above, when an electrosurgical instrument is activated a high frequency current (which is referred to herein as a driving electrosurgical signal) is provided to a live or active electrode of the electrosurgical instrument which passes through the patient and returns via a return electrode. When the electrosurgical instrument is a monopolar electrosurgical instrument the return electrode is separate from the instrument and is placed on the patient. When the electrosurgical instrument is a bipolar electrosurgical instrument, the return electrode forms part of the electrosurgical instrument and the current passes through the patient from the active electrode of the electrosurgical instrument to the return electrode of the electrosurgical instrument.

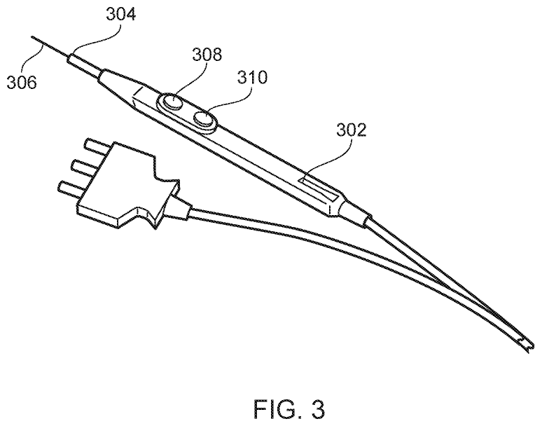

When there are multiple electrosurgical instruments present at a surgical site simultaneously there is a possibility that when one of the electrosurgical instruments is activated or energized with a driving electrosurgical signal that at least some of the return signal or current is capacitively coupled or leaked to the active electrode and/or the return electrode of the other instrument. In such cases, at least part of the return current is not travelling through the intended path (i.e., via the appropriate return electrode) thus there is a possibility of harm to the patient. This is most likely to occur when the instrument being activated is a monopolar instrument and the other instrument is a bipolar instrument as the current from the active electrode instrument may not travel to the return pad as intended, but instead travel to the return electrode of the bipolar instrument. However, this may occur between any pair of electrosurgical instruments. Specifically, this capacitive coupling may occur even if the second instrument does not have a built-in return path (e.g. is a monopolar instrument) if there is sufficient capacitance between the electrosurgical instrument and the electrosurgical generator. This problem is particularly prevalent in surgical robot systems since surgical robot systems tend to have longer cables between the electrosurgical instruments and the corresponding electrosurgical generator, creating more capacitance therebetween, than in a manual set-up such as that described in FIGS. 3-6. The surgical robot itself may also add capacitance.

Accordingly, described herein are electrosurgical connection units for a surgical robot arm that comprise, in addition to one or more activation switch units which can be selectively activated to cause an activation signal to be output on the input port, one or more connection switch units which can selectively connect the output port to the input port. The one or more connection switch units allow for an electrosurgical instrument attached to a robot arm to be connected to the electrosurgical generator (and the related long cables) only when the electrosurgical instrument is to be activated, and disconnected from the electrosurgical generator otherwise. In other words, when the electrosurgical instrument is not in use it is electrically isolated from the rest of the system (e.g. the electrosurgical generator and the cables therebetween). When the electrosurgical instrument is disconnected from the electrosurgical generator the extra current path through the electrosurgical instrument for current applied to another electrosurgical instrument is eliminated or does not exist.

Since it may be dangerous to connect and/or disconnect the input port from the output port when an electrosurgical generator is providing a driving electrosurgical signal to the input port, it is desirable that the electrosurgical connection unit is configured to activate the connection switch unit prior to activating an activation switch unit so that a driving electrosurgical signal generated in response to an activation signal will arrive at the input port after the input port has be electrically connected to the output port. Similarly, it is desirable that the electrosurgical connection unit is configured to deactivate the connection switch unit after deactivating an activation switch unit so that the input port ceases to receive a driving electrosurgical signal prior to the input port being disconnected from the output port.

As described in more detail below, the timing of the activation and deactivation of the connection switch unit and the appropriate activation switch unit may be implemented in hardware or software. For example, in some cases the control unit may be configured to, in response to receiving a control signal indicating the electrosurgical instrument attached to the arm is to be activated or energised, generate or invoke switch control signals to the different switch units at different times. In other examples, the control unit may be configured to, in response to receiving a control signal indicating that the electrosurgical instrument attached to the arm is to be activated or energised, generate or invoke the same switch control signal at the same time to the different switch units, and there may be one or more hardware units or circuits between the control unit and the switch units which cause, in response to the same switch control unit signal, the connection switch unit to be activated prior to, and deactivated after, an activation switch unit. Implementing the timing of the activation and deactivation of the connection switch units and the appropriate activation switch unit in hardware may be safer.

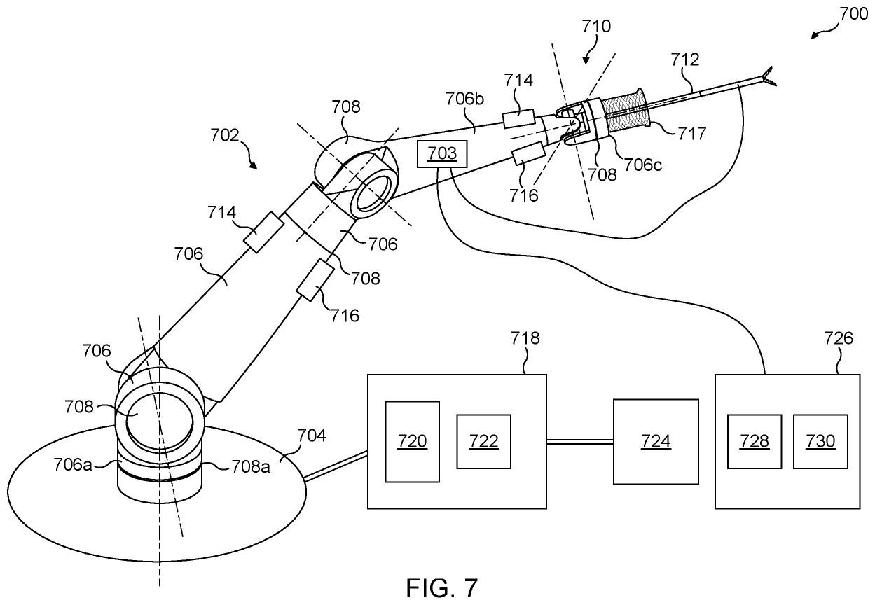

Reference is now made to FIG. 7 which shows an example surgical robot system 700 in which the electrosurgical connection units described herein may be implemented. The surgical robot system 700 comprises a robot arm 702 which comprises an electrosurgical connection unit 703 for connecting an electrosurgical instrument attached to the arm to an electrosurgical generator.

The robot arm 702 extends from a proximal end attached to a base 704. The robot arm 702 comprises a number of rigid links 706. The links are coupled by revolute joints 708. The most proximal link 706a is coupled to the base by joint 708a. It and the other links are coupled in series by further ones of the joints 708. Suitably, a wrist 710 is made up of four individual revolute joints. The wrist 710 couples one link 706b to the most distal link 706c of the arm. The most distal link 706c is at the distal end of the arm and carries an attachment structure 717 for a surgical instrument 712. Each joint 708 of the robot arm 702 has one or more motors 714 which can be operated to cause rotational motion at the respective joint, and one or more position and/or torque sensors 716 which provide information regarding the current configuration and/or load at that joint. The motors may be arranged proximally of the joints whose motion they drive, so as to improve weight distribution. For clarity, only some of the motors and sensors are shown in FIG. 7. The arm may be generally as described in our patent application PCT/GB2014/053523.

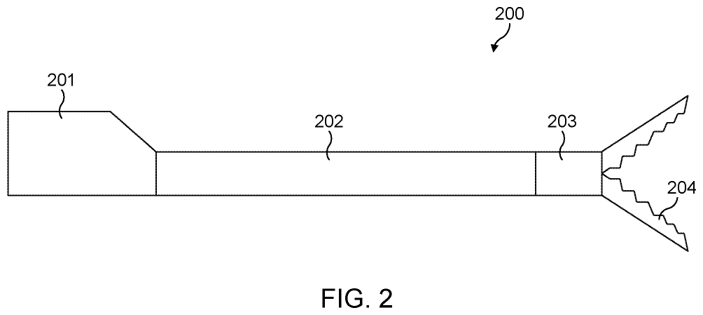

The arm terminates in an attachment structure 717 for interfacing with the instrument 712. The instrument 712 may take the form described with respect to FIG. 2. The attachment structure 717 comprises a drive assembly for driving articulation of the instrument. Movable interface elements of the drive assembly interface mechanically engage corresponding movable interface elements of the instrument interface in order to transfer drive from the robot arm to the instrument. One instrument is exchanged for another several times during a typical operation. Thus, the instrument is attachable and detachable from the robot arm during the operation. Features of the drive assembly interface and the instrument interface aid their alignment when brought into engagement with each other, so as to reduce the accuracy with which they need to be aligned by the user.

The instrument 712 comprises an end effector for performing an operation. The end effector may take any suitable form. For example, the end effector may be smooth jaws, serrated jaws, a gripper, a pair of shears, a needle for suturing, a camera, a laser, a knife, a stapler, a cauteriser, a suctioner.

A variety of instrument types are known, each adapted to perform a particular surgical function. One example type of instrument is an electrosurgical instrument which is adapted to perform an electrosurgical function. As described above, electrosurgery is the passing of a high frequency (i.e. radio frequency) current through tissue to cause a desired effect (e.g. cutting the tissue or coagulating the tissue). There are two types of electrosurgery monopolar and bipolar. In monopolar electrosurgery the high frequency current passes through the patient from a live or active electrode of the electrosurgical instrument to a separate return electrode placed on the patient, which may also be referred to as a dispersive electrode pad, a grounding pad, a neutral electrode, a grounding mat, an indifferent electrode, or a patient electrode. In bipolar electrosurgery the active and return electrodes are both within the electrosurgical instrument and the current passes through the patient from the active electrode of the electrosurgical instrument to the return electrode of the electrosurgical instrument. An electrosurgical instrument which is configured for monopolar electrosurgery (e.g. an electrosurgical instrument that comprises an active electrode only) will be referred to herein as a monopolar electrosurgical instrument, and an electrosurgical instrument which is configured for bipolar electrosurgery (e.g. an electrosurgical instrument that comprises both an active electrode and a return electrode) will be referred to herein as a bipolar electrosurgical instrument.

As described with respect to FIG. 2 the instrument comprises an articulation between the instrument shaft and the end effector. The articulation comprises several joints which permit the end effector to move relative to the shaft of the instrument. The joints in the articulation are actuated by driving elements, such as cables. These driving elements are secured at the other end of the instrument shaft to the interface elements of the instrument interface. Thus, the robot arm transfers drive to the end effector as follows: movement of a drive assembly interface element moves an instrument interface element which moves a driving element which moves a joint of the articulation which moves the end effector.

Controllers for the motors, torque sensors and encoders are distributed with the robot arm. The controllers are connected via a communication bus to a robot control unit 718. The robot control unit 718 comprises a processor 720 and a memory 722. Memory 722 stores in a non-transient way software that is executable by the processor 720 to control the operation of the motors 714 to cause the robot arm 702 to operate in the manner described herein. In particular, the software can control the processor 720 to cause the motors (for example via distributed controllers) to drive in dependence on inputs from the sensors 716 and from a surgeon command interface 724. The robot control unit 718 is coupled to the motors 714 for driving them in accordance with outputs generated by execution of the software. The robot control unit 718 is coupled to the sensors 716 for receiving sensed input from the sensors, and to the command interface 724 for receiving input from it. The respective couplings may, for example, each be electrical or optical cables, or may be provided by a wireless connection. The command interface 724 comprises one or more input devices whereby a user can request motion of the end effector in a desired way. The input devices could, for example, be manually operable mechanical input devices such as hand controllers or joysticks, or contactless input devices such as optical gesture sensors. The software stored in memory 722 is configured to respond to those inputs and cause the joints of the arm and instrument to move accordingly, in compliance with a pre-determined control strategy. The control strategy may include safety features which moderate the motion of the arm and instrument in response to command inputs. Thus, in summary, a surgeon at the command interface 724 can control the instrument 712 to move in such a way as to perform a desired surgical procedure. The robot control unit 718 and/or the command interface 724 may be remote from the robot arm 702.

The robot arm 702 also comprises an electrosurgical connection unit 703 for connecting an electrosurgical instrument 712 attached to the arm to an electrosurgical generator 726. As described above, electrosurgical instruments are driven by a high frequency current which may be referred to herein as a driving electrosurgical signal. The driving electrosurgical signals are generated by an electrosurgical generator 726, which may also be referred to as an electrosurgery generator, electrosurgical end unit, electrosurgery end unit, or ESU. Electrosurgical generators are generally capable of generating multiple different current waveforms to achieve different surgical effects. For example, many standard electrosurgical generators can be configured to generate COAG, CUT and BLEND waveforms. The COAG waveform consists of bursts of radio frequency, which when used at a low power setting causes a desiccation effect, and when used at a high-power setting causes a fulguration effect. The CUT waveform is a continuous waveform at a lower voltage, but higher current than COAG, which causes the tissue to be cut. A BLEND waveform is essentially a CUT waveform with a lower duty cycle. For example, the duty cycle of a CUT waveform is typically between 15% and 75%, whereas a CUT waveform typically has a duty cycle greater than 75%. The off time allows the tissue to cool creating some haemostasis. Accordingly, a BLEND waveform is typically used where haemostasis is required as tissue is cut. It will be evident to a person of skill in the art that these are examples only and that different electrosurgical generators may be configured to generate different and/or additional waveforms.

The electrosurgical generator 726 comprises any suitable means for configuring the waveforms that can be generated. For example, an electrosurgical generator 726 may comprise a user interface that comprises, for example, switches, buttons, dials etc., which enable a user to configure each supported waveform (e.g. a CUT waveform, a COAG waveform and a BLEND waveform). In other examples, the electrosurgical generator 726 (and the individual waveforms) may be configured electronically, such as, but not limited to, via a control signal (or a set of control signals) transmitted to the electrosurgical generator from a computing device. For example, the electrosurgical generator 726 may be connected, via, for example, a communications network, such as, but not limited to an Ethernet or a Wi-Fi communications network, to the robot control unit 718 and the waveforms may be configured from the command interface 724. The user may be able to configure, for example, the voltage and/or frequency of the waveform.

The electrosurgical generator 726 also comprises control logic 728 which is configured to receive activation signals indicating which waveform of the plurality of supported waveforms are to be activated by the electrosurgical generator 726. For example, where the electrosurgical generator 726 can generate a driving electrosurgical signal with a CUT waveform or a driving electrosurgical signal with a COAG waveform the electrosurgical generator 726 may be configured to receive one or more activation signals indicating which of the CUT waveform and the COAG waveform is to be used to generate the driving electrosurgical signal. In response to the control logic 728, detecting an activation signal indicating that a driving electrosurgical signal with a CUT waveform is to be activated the electrosurgical generator 726 (e.g. the RF generation logic 730) outputs a driving electrosurgical signal with a CUT waveform (as previously configured). Similarly, in response to the control logic 728 detecting an activation signal indicating that a driving electrosurgical signal with a COAG waveform is to be activated the electrosurgical generator 726 (e.g. the RF generation logic 730) outputs a driving electrosurgical signal with a COAG waveform (as previously configured). In some cases, the electrosurgical generator 726 may be configured to continue outputting a driving electrosurgical signal with the desired waveform so long as it detects the corresponding activation signal (and a fault condition has not been detected), and to cease outputting a driving electrosurgical signal with the desired waveform as soon as it ceases to detect the corresponding activation signal.

When an activation signal is detected by the control logic 728, in addition to causing a driving electrosurgical signal with the desired waveform to be output, the control logic 728 may cause a feedback signal to be output to alert the user of the activation of a particular waveform. The feedback may be in the form of visual feedback (e.g. an indicator light on a display panel of the electrosurgical generator 726) or audible feedback (e.g. a tone).

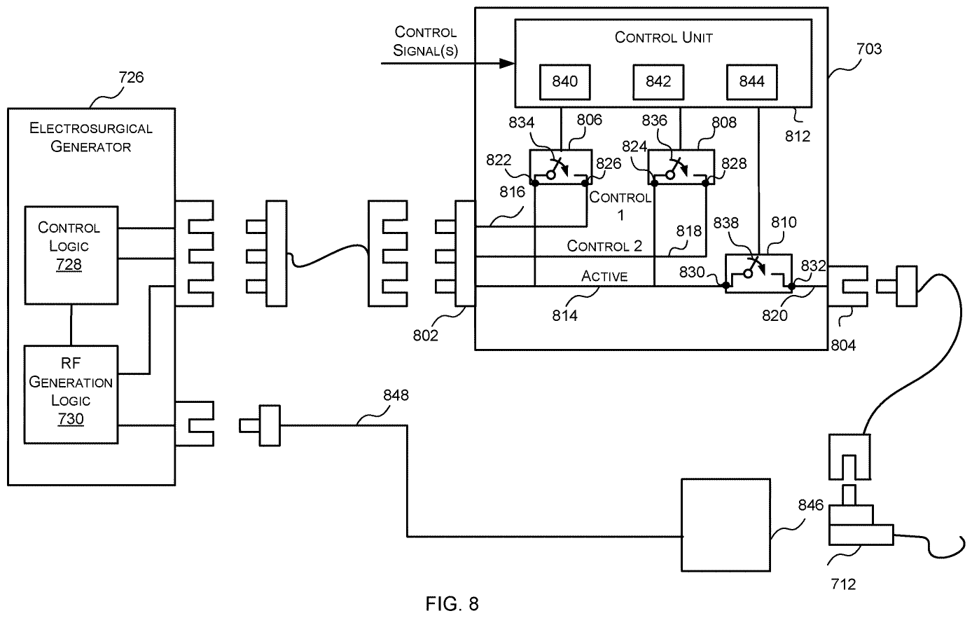

The electrosurgical connection unit 703 is configured to act as an intermediary between an electrosurgical instrument 712 attached to the robot arm 702 and an electrosurgical generator 726. Specifically, the electrosurgical connection unit 703 is configured to, in response to receiving one or more control signals from an external computing device indicating that the electrosurgical instrument 712 attached to the robot arm 702 is to be activated with a particular waveform, (i) electrically connect the electrosurgical generator 726 to the electrosurgical instrument 712; and (ii) transmit one or more activation signals to the electrosurgical generator which cause the electrosurgical generator 726 to output a driving electrosurgical signal with the particular waveform. The one or more control signals received by the electrosurgical connection unit 703 may be generated by, for example, the robot control unit 718 in response to the surgeon or other user providing input via the command interface 724 indicating that the electrosurgical instrument attached to the arm currently being controlled is to be driven by a driving electrosurgical signal with a desired waveform. The electrosurgical connection unit 703 is also configured to receive any driving electrosurgical signal produced by the electrosurgical generator in response to an activation signal and provide the received driving electrosurgical signal to the electrosurgical instrument attached to the arm. Example electrosurgical connection units 703 are described below with respect to FIGS. 8-13.

The electrosurgical connection unit 703 may be integral with the robot arm 702 or may be removably attached to the robot arm 702. The electrosurgical connection unit 703 may be removably attached to the robot arm 702 using any suitable means such as, but not limited to, Velcro™, or gaffer tape. Although the electrosurgical connection unit 703 is shown in FIG. 7 as being attached to a middle link 706b of the robot arm 702, the electrosurgical connection unit 703 may be attached to any suitable part of the robot arm 702. For example, the electrosurgical connection unit 703 may be connected to any link 706a, 706b, 706c of the robot arm 702 or the electrosurgical connection unit 703 may be connected to the base 704 of the robot arm 702. In some cases the base 704 may comprise or be attached to a cart and the electrosurgical connection unit may be connected to the cart. The robot arm 702 is typically covered in a sterile drape during surgery. Where there is an opening in the drape around the base 704 of the robot arm 702, attaching the electrosurgical connection unit 703 to the base 704 or the most proximal link 706a may make it easier to connect the electrosurgical connection unit 703 to the electrosurgical generator 726 and/or the electrosurgical instrument 712 via the opening in the drape. In some cases, components of the electrosurgical connection unit 703 may be attached to different parts of the robot arm 702. For example, as described below, the electrosurgical connection unit may comprise an input port, an output port, one or more activation switch units, a connection switch unit and a control unit. In some cases, the control unit may be situated in a different part of the arm from the input port, output port, activation switch units, and connection switch unit. Although the electrosurgical connection unit 703 is shown in FIG. 7 as part of, or attached to, the robot arm 702, in other examples, the electrosurgical connection unit 703 may be integral with the robot control unit 718. However, this may increase the cable length between the electrosurgical connection unit 703 and the electrosurgical instrument 712 which may increase the capacitance between the electrosurgical connection unit 703 and the electrosurgical instrument 712 and affect the tidiness of the operating room.

Reference is now made to FIG. 8 which illustrates an example electrosurgical connection unit 703 for connecting a monopolar electrosurgical instrument to an electrosurgical generator 726. The electrosurgical connection unit 703 comprises an input port 802, an output port 804, a plurality of activation switch units 806, 808, a connection switch unit 810 and a control unit 812.

The input port 802 is connectable (directly or indirectly) to an electrosurgical generator 726 and is configured to receive a driving electrosurgical signal generated by the electrosurgical generator 726 and to transmit or output one or more activation signals to the electrosurgical generator 726. Each activation signal indicates to the electrosurgical generator 726 that a driving electrosurgical signal with a desired waveform of the plurality of waveforms supported by the electrosurgical generator 726 is to be activated. The input port 802 may be electrically coupled to a plurality of wires or conductors-an input active wire or conductor 814 to carry the driving electrosurgical signal received on the input port 802 (e.g. from the electrosurgical generator 726) and one or more control wires or conductors 816, 818 to carry the activation signals(s) to be output on the input port 802 (e.g. to be sent to the electrosurgical generator 726). In the example shown in FIG. 8, there are two control wires 816, 818, and one control wire 816 is configured to carry a first activation signal to the input port 802 (to ultimately be transmitted to the electrosurgical generator 726) that indicates that a driving electrosurgical signal with a first waveform (e.g. a CUT waveform) is to be activated and the other control wire 818 is configured to carry a second activation signal to the input port 802 (to ultimately be transmitted to the electrosurgical generator 726) that indicates that a driving electrosurgical signal with a second waveform (e.g. a COAG waveform) is to be activated. However, it will be evident to a person of skill in the art that this is an example only and that there may be fewer than two control wires and/or more than two control wires for carrying activation signal(s) to the input port 802.

The input port 802 may be configured to receive one or more cables over which the driving electrosurgical signal is received from the electrosurgical generator 726 and the activation signals are transmitted to the electrosurgical generator 726. In some cases, the input port 802 may be configured to receive a single cable over which the driving electrosurgical signal and the activation signals are transmitted. In other cases, the input port 802 may be configured to receive a plurality of cables over which the driving electrosurgical signal and the activation signals are transmitted. For example, there may be one cable per signal. In some cases, the input port 802 may comprise one connector for each expected cable that is configured to engage a corresponding connector of the cable. In some cases, the connector(s) of the input port 802 may be male connectors which are configured to receive a corresponding female connector of a cable connected directly or indirectly to the electrosurgical generator 726.

The output port 804 is connectable (directly or indirectly) to an electrosurgical instrument 712 attached to the robot arm 702. The output port 804 is electrically connected or coupled to an output active wire 820 which is configured to provide a driving electrosurgical signal received on the input port 802 to the output port 804.

The output port 804 may be configured to receive a cable over which the driving electrosurgical signal output from the output port 804 is transmitted to the electrosurgical instrument 712. In some examples, the output port 804 comprises a female connector configured to engage a corresponding male connector connected to a cable which is connected directly or indirectly to the electrosurgical instrument 712.

Each activation switch unit 806, 808 is configured to, when activated, cause an activation signal to be transmitted to the input port 802 to be output thereby. When the input port 802 is connected to an electrosurgical generator this provides the activation signal to the electrosurgical generator 726. The activation signal indicates that a driving electrosurgical signal with a desired waveform, of the plurality of waveforms supported by the electrosurgical generator 726, is to be activated or generated. In response to detecting an activation signal, the electrosurgical generator 726 outputs a driving electrosurgical signal with the desired waveform. When the input port 802 is connected to an electrosurgical generator the input port 802 receives the driving electrosurgical signal with the desired waveform. The driving electrosurgical signal is then carried through the electrosurgical connection unit 703 on the input active wire 814.

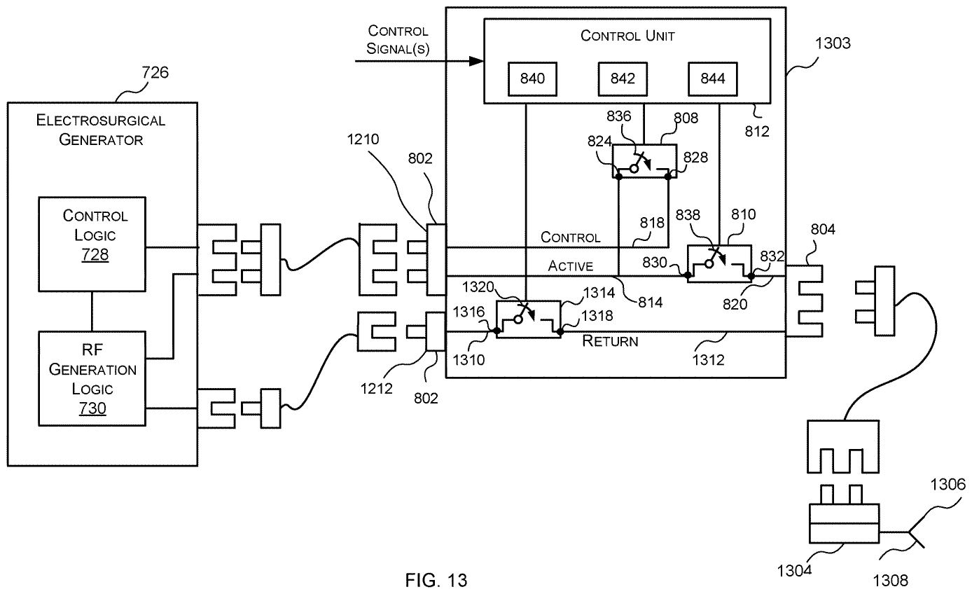

In the example of FIG. 8 there are two activation switch units 806 and 808. When the first activation switch unit 806 is activated, a first activation signal is transmitted over the first control wire 816 to the input port 802 for output. When the input port 802 is connected to an electrosurgical generator this provides the first activation signal to the electrosurgical generator 726. The first activation signal indicates to the electrosurgical generator 726 that a driving electrosurgical signal with a first desired waveform (e.g. CUT waveform) is to be activated or output. In response to detecting the first activation signal, the electrosurgical generator 726 generates and outputs a driving electrosurgical signal with the desired waveform (e.g. CUT waveform). When the second activation switch unit 808 is activated, a second activation signal is transmitted over the second control wire 818 to the input port 802 for output. When the input port 802 is connected to an electrosurgical generator 726, this provides the second activation signal to the electrosurgical generator 726. The second activation signal indicates to the electrosurgical generator 726 that a driving electrosurgical signal with a second desired waveform (e.g. COAG waveform) is to be activated or output. In response to detecting the second activation signal, the electrosurgical generator 726 generates and outputs a driving electrosurgical signal with the second desired waveform (e.g. COAG waveform). However, it will be evident to a person of skill in the art that this is an example only and that there may be more than two activation switch units or only one activation switch unit (see, for example, FIG. 13).

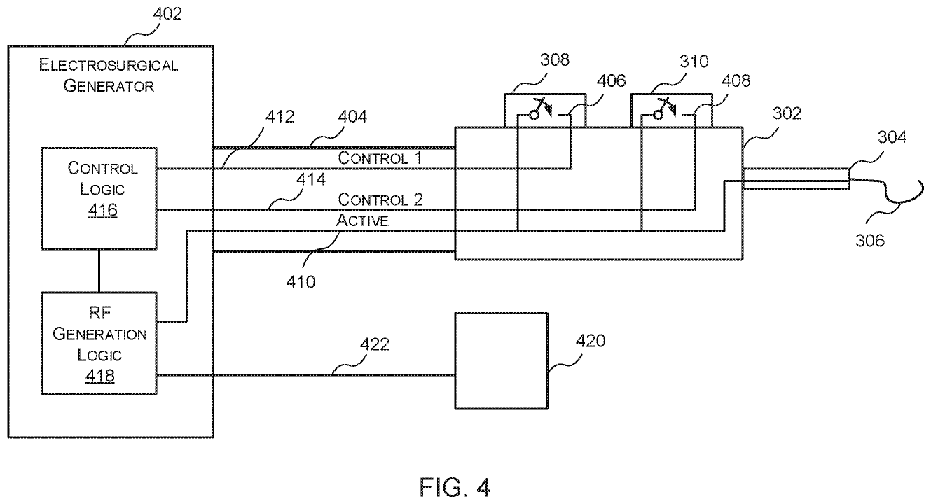

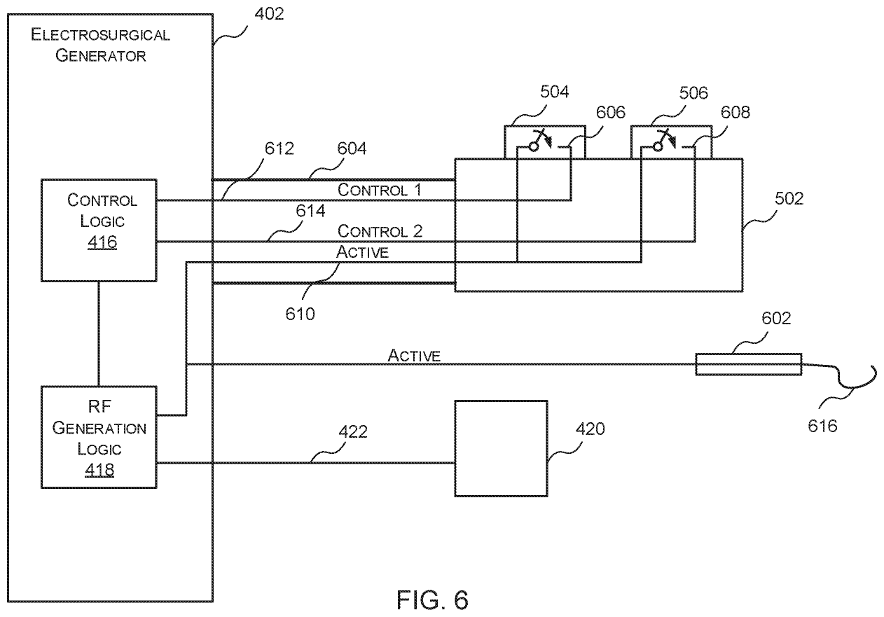

In the example shown in FIG. 8, a first port 822, 824 of each activation switch unit 806, 808 is connected to the input active wire 814 and a second port 826, 828 of each activation switch unit 806, 808 is connected to one of the control wires 816, 818. Specifically, the second port 826 of the first activation switch unit 806 is connected to the first control wire 816, and the second port 828 of the second activation switch unit 808 is connected to the second control wire 818. In this example, when an activation switch unit 806, 808 is activated the input active wire 814 is electrically connected to the corresponding control wire 816, 818 (i.e. the input active wire 814 is shorted to the corresponding control wire 816, 818) which causes an activation signal to be transmitted on that control wire 816, 818. When an electrosurgical generator is electrically connected to the input port 802, this causes an activation signal to be transmitted to the electrosurgical generator 726. Specifically, when an electrosurgical generator is electrically connected to the input port 802, activation of an activation switch unit 806 808 closes a control loop extending between the electrosurgical generator 726 and the electrosurgical connection unit 703 which can be detected by the electrosurgical generator 726 (e.g. control logic 728 of the electrosurgical generator 726). When the electrosurgical connection unit 703 is configured to generate the activation signals in this manner the electrosurgical connection unit 703 can be connected to existing electrosurgical generators, such as those described above with reference to FIGS. 4 and 6, which are configured to detect activation signals by detecting a closure of a control loop.

The connection switch unit 810 is configured to, when activated, cause at least a portion of the input port 802 to be electrically connected to the output port 804 such that any driving electrosurgical signal received on the input port 802 (e.g. in response to an activation signal being transmitted to the electrosurgical generator 726) is output on the output port 804.

In the example shown in FIG. 8, a first port 830 of the connection switch unit 810 is connected to the input active wire 814 and a second port 832 of the connection switch unit 810 is connected to the output active wire 820. In this example, when the connection switch unit 810 is activated, the input active wire 814 is electrically connected to the output active wire 820 (i.e. the input active wire 814 is shorted to the output active wire 820) which causes a driving electrosurgical signal received on the input port 802 to be output on the output port 804. As described above, the connection switch unit 810 allows the active path between an electrosurgical instrument attached to the electrosurgical connection unit 703 and an electrosurgical generator attached to the electrosurgical connection unit 703 to be disconnected or disabled while the instrument is not in use. This reduces the chances, when the electrosurgical instrument is near a surgical site when another electrosurgical instrument is energised, of the return energy taking the active path through the electrosurgical instrument instead of the appropriate return path.

Each switch unit 806, 808, 810 comprises at least one switch 834, 836, 838 connected in series with the first port and the second port of the switch unit 806, 808, 810. When a switch unit 806, 808, 810 is activated all the switch(es) 834, 836, 838 of the switch unit 806, 808, 810 are placed in the closed position so as to connect the first and second ports 822, 824, 826, 828, 830, 832 of the switch unit 806, 808, 810. In the example of FIG. 8 each switch unit 806, 808, 810 comprises one switch 834, 836, 838. However, in other examples, one or more of the switch units 806, 808, 810 may comprise a plurality of switches in series.

Having multiple switches in series in an activation switch unit 806, 808 prevents an activation signal inadvertently being transmitted by the electrosurgical connection unit 703 to the electrosurgical generator (causing the electrosurgical generator to inadvertently output a driving electrosurgical signal) when one of the switches fails in the closed position. Causing a driving electrosurgical signal instrument to be inadvertently generated could be extremely dangerous. Similarly, having multiple switches in series in a connection switch unit 810 prevents the electrosurgical generator from inadvertently being connected to the electrosurgical instrument when one of the switches fails in the closed position. Each switch may be implemented, for example, by a relay, such as an electromechanical relay (EMR) or a solid-state relay (SSR). As is known to those of skill in the art, in electromechanical relays (EMR), contacts are opened or closed by a magnetic force. With solid-state relays (SSR), there are no contacts and switching is electronic.

The control unit 812 is configured to control the activation switch units 806, 808 and the connection switch unit 810 in response to control signals received from an external computing device. As described above, for an electrosurgical instrument attached to the robot arm 702 to be activated by a driving electrosurgical signal one of the activation switch units are activated to cause the electrosurgical generate to generate a driving electrosurgical signal and the connection switch unit is activated so that any driving electrosurgical signal received from the electrosurgical generator is provided to the electrosurgical instrument. However, as described above, it is desirable that the connection switch unit be activated prior to the relevant activation switch unit so that the connection switch unit is not changing state while a driving electrosurgical signal is being output from the electrosurgical generator. Accordingly, the control unit 812 is configured to, in response to receiving one or more control signals from an external computing device indicating that the electrosurgical instrument attached to the robot arm 702 is to be activated or energised with a desired waveform, cause (i) the connection switch unit 810 to be activated, and (ii) one of the activation switch units 806, 808 to be activated subsequent to activation of the connection switch unit 810. When the input port 802 is connected to an electrosurgical generator 726 and the output port 804 is connected to an electrosurgical instrument 712 attached to the arm, this causes the electrosurgical instrument to be activated or energised by a driving electrosurgical signal with the desired waveform.

The timing of the activation and deactivation of the connection switch unit and the appropriate or relevant activation switch unit may be implemented in hardware or software. For example, in some cases the control unit 812 may be configured to, in response to receiving a control signal indicating that the electrosurgical instrument attached to the arm is to be activated or energised, generate or invoke a first switch control signal indicating that the connection switch unit is to be activated and subsequently generate or invoke a second switch control signal indicating that the appropriate activation switch unit is to be activated.

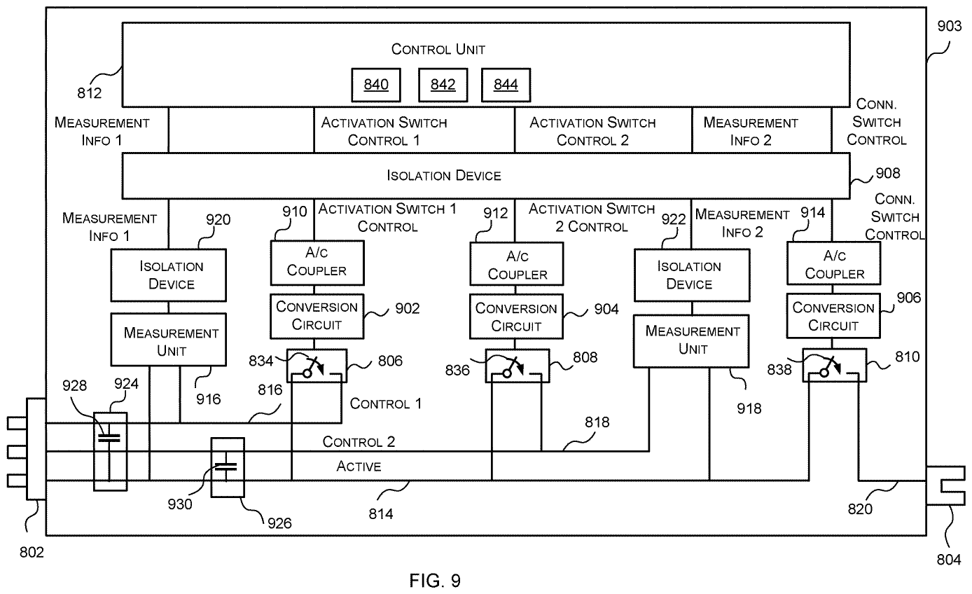

However, in other examples, the electrosurgical connection unit 703, 903, 1203, 1303 may comprise a hardware unit or circuit for each switch unit that is situated between the control unit and that switch unit. Each such hardware unit or circuit is configured to receive the switch control signal from the control unit 812 for the corresponding switch unit and activate (and deactivate) the switch unit at the correct time. In particular, if the hardware unit for the connection switch unit and the hardware unit for the activation switch unit receive the same switch control signal, the hardware unit for the connection switch unit will activate the connection switch unit prior to the hardware unit for the activation switch unit activating the activation switch unit; and the hardware unit for the connection switch unit will deactivate the connection switch unit after the hardware unit for the activation switch unit deactivates the activation switch unit. In these cases, the control unit 812 may be configured to, in response to receiving a control signal indicating that the electrosurgical instrument attached to the robot arm is to be activated, invoke or generate, at the same time, a first control signal indicating that the connection switch unit is to be activated and a second switch control signal indicating that the appropriate activation switch unit is to be activated—and then the hardware units or circuits will take care of the timing of the activations (and deactivations) of the connection switch unit and the activation switch unit. Implementing the timing of the activation and deactivation of the connection switch unit and the appropriate activation switch unit in hardware, vs software, may be safer (e.g. less prone to error). Example hardware units which may be used to implement the desired switch unit activation timing are described below with respect to FIG. 9.

The control signals received by the control unit 812 may be generated by an external computing device in response to input received from a surgeon or another user indicating that the electrosurgical instrument attached to a particular arm is to be activated by a driving electrosurgical signal with a particular waveform. In some cases, the control signals may be generated by the robot control unit 718 in response to input received from the surgeon or another user via the command interface 724 indicating that the electrosurgical instrument attached to a particular arm is to be activated by a driving electrosurgical signal with a particular waveform.

In these cases, the command interface 724 may comprise one or more input devices that allow the user to indicate that an electrosurgical instrument that a user is currently controlling is to be activated by a driving control signal and what type of waveform. For example, where the command interface 724 comprises manually operable input devices such as hand controllers or joysticks, the hand controllers or joysticks may comprise one or more buttons, switches, or the like that allow the user to indicate that the electrosurgical instrument that is currently being controlled is to be activated by a driving electrosurgical signal and the type of waveform. For example, the hand controllers or joysticks may comprise a CUT button and a COAG button which the user can press to indicate that the electrosurgical instrument is to be activated by a CUT waveform or a COAG waveform. In some cases, to avoid a driving electrosurgical signal from being transmitted to an electrosurgical instrument by inadvertent contact with such buttons or switches, the one or more buttons or switches may only be able to cause a driving electrosurgical signal to be transmitted to the electrosurgical generator if the robot control unit detects that a user is currently grasping the hand controllers or joysticks.

In other examples, when the user is controlling an electrosurgical instrument the user may be provided with one or more options on a graphical user interface displayed on a display screen that can be clicked, or otherwise selected, by the user to indicate that the electrosurgical instrument is to be activated and the type of waveform the electrosurgical instrument is to be activated with. For example, a CUT button and a COAG button may be displayed on a display screen that can be clicked, or otherwise selected, by the user to indicate that the electrosurgical instrument is to be activated with a CUT waveform or a COAG waveform.

In yet other examples, the command interface 724 may comprise a combination of the buttons and graphical user interface components described above to allow the user to indicate that a particular electrosurgical instrument is to be activated and the waveform to be used for the driving electrosurgical signal. For example, the user interface may allow the surgeon, or other user, to indicate the type of waveform to be used for the driving electrosurgical signal and the electrosurgical instrument to be activated, and the hand controller or joysticks may comprise a single button which, when depressed, indicates that the selected electrosurgical instrument is to be activated with a driving electrosurgical signal with the selected waveform. In some cases, the hand controller or joystick may comprise one or more coloured LEDs near the activation button which indicates the selected waveform (e.g. a blue LED may be illuminated when a COAG waveform is selected, and a yellow LED may be illuminated when CUT waveform is selected).

Thus, in addition to a surgeon being able to control the movement of an instrument 712 attached to a robot arm 702 via the command interface 724, when that instrument is an electrosurgical instrument, the surgeon may also be able to control, from the command interface 724, when that electrosurgical instrument 712 is activated or energised and the type of waveform of the driving electrosurgical signal in a safe and secure manner.

The control unit 812 may comprise a communications module 840, one or more processors 842 and/or a memory 844. The communications module 840 is configured to receive control signals from the external computing device (e.g. robot control unit 718). The communications module 840 may be configured to receive the control signals from the external computing device (e.g. robot control unit 718) in any suitable manner such as, but not limited to, electrically, optically or wirelessly. For example, in some cases, the communications module 840 may be coupled to a wired communication network, such as, but not limited to, an Ethernet network, over which the communications module 840 receives control signals from the external computing device (e.g. robot control unit 718). In other cases, the communications module 840 may be coupled to a wireless communication network, such as, but not limited to, a Wi-FI™ network or an NFC (Near Field Communication) network, over which the communications module 840 receives the control signals from the external computing device (e.g. robot control unit 718).

In some cases, in addition to being able to receive control signals from the external computing device (e.g. robot control unit 718) the communications module 840 may also be able to transmit data or information to the external computing device (e.g. robot control unit 718). For example, as described in more detail below, the electrosurgical connection unit 703 may also comprise an impedance measurement unit which is configured to measure the impedance across the activation switch units 806, 808 and information related to the detected impedance(s) may be transmitted to the external computing device (e.g. robot control unit 718) via the communications module 840. Where the communications module 840 can receive information from, and transmit information to, the external computing device (e.g. robot control unit 718) the communications module 840 may be described as a transceiver.

The memory 844 is configured to store computer-executable instructions that when executed by the one or more processors 842 cause the one or more processors 842 to perform the functions described herein. Specifically, the one or more processors 842 are configured (by the computer-executable instructions) to analyse any control signal received by the communications module 840 and can cause activation of the connection switch unit 810 and one of the activation switch units 806, 808 based on the analysis. The control signals are configured to indicate to the one or more processors 842 when an electrosurgical instrument is to be activated or energised by a driving electrosurgical signal and the waveform of the driving electrosurgical signal. Both the activation information and the waveform information may be included in a single control signal, or the activation information and the waveform information may be included in different control signals (e.g. there may be a control signal that indicates that the electrosurgical instrument is to be activated or energised and a different control signal that indicates that waveform of the driving electrosurgical signal). The control signals may take any suitable form that is understood by the one or more processors 842. In some cases, as described in more detail below, the control signals may be tokens.

The one or more processors 842 are configured to analyse any control signal, or set of control signals, received by the communications module 840 to determine whether the electrosurgical instrument attached to the arm is to be activated and if the electrosurgical instrument attached to the arm is to be activated the desired waveform of the driving electrosurgical signal. In response to determining from a received control signal, or set of control signals, that the electrosurgical instrument is to be activated by a driving electrosurgical signal with a particular waveform the one or more processors 842 may be configured to cause (i) the connection switch unit 810 to be activated and (ii) the activation switch unit 806, 808 associated with the identified waveform to be activated, subsequent to activation of the connection switch unit 810.

For example, where there are two activation switch units 806, 808 and one activation switch unit 806 is configured to cause a first activation signal to be transmitted to the electrosurgical generator which indicates that the electrosurgical instrument is to be activated by a driving electrosurgical signal with a first waveform (e.g. CUT waveform), and the other activation switch unit 808 is configured to cause a second activation signal to be transmitted to the electrosurgical generator which indicates that the electrosurgical instrument is to be activated by a driving electrosurgical signal with a second waveform (e.g. COAG waveform), if the one or more processors 842 determine from a received control signal, or set of control signals, that the electrosurgical instrument attached to the arm is to be activated by a driving electrosurgical signal with the first waveform (e.g. CUT waveform) the one or more processors may be configured to cause the first activation switch unit 806 to be activated (subsequent to activation of the connection switch unit 810), and if the one or more processors 842 determine from a received control signal, or set of control signals, that the electrosurgical instrument is to be activated by a driving electrosurgical signal with the second waveform (e.g. COAG waveform) the one or more processors 842 may be configured to cause the second activation switch unit 808 to be activated (subsequent to activation of the connection switch unit 810).

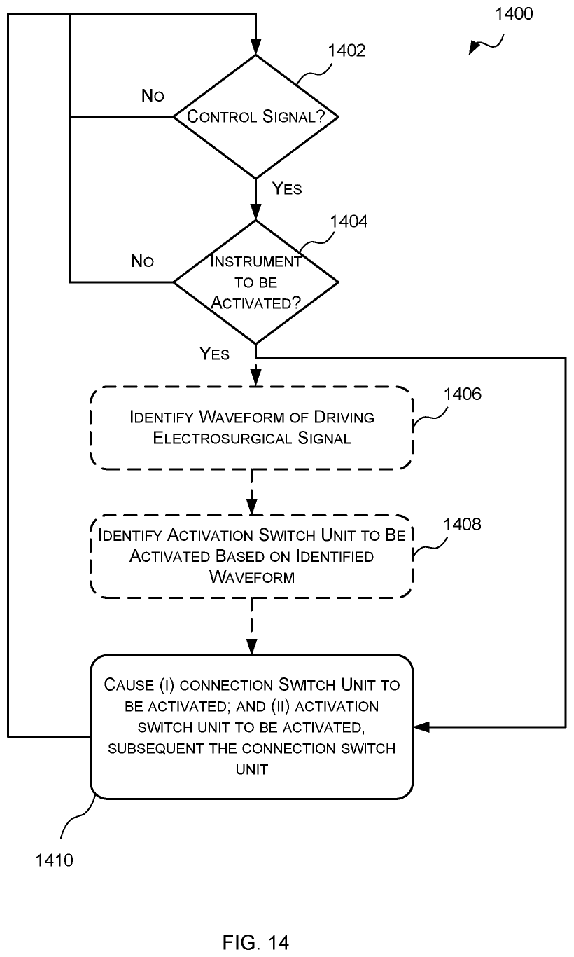

In some cases, the one or more processors 842 may be configured to cause a particular switch unit 806, 808, 810 to be activated by outputting one or more signals that cause all the switches 834, 836, 838 of that switch unit 806, 808, 810 to be in a closed position. An example method for processing control signals received from an external computing device, which may be implemented by the one or more processors 842, is described below with respect to FIG. 14.

In the example shown in FIG. 8, a separate return electrode 846 is directly connected to the electrosurgical generator 726 via a separate cable 848.

Reference is now made to FIG. 9 which illustrates a second example electrosurgical connection unit 903 for connecting a monopolar electrosurgical instrument to an electrosurgical generator 726. The example electrosurgical connection unit 903 of FIG. 9 is the same as the electrosurgical connection unit 703 of FIG. 8 except the electrosurgical connection unit 903 includes one or more further optional components. Specifically, like the electrosurgical connection unit 703 of FIG. 8, the electrosurgical connection unit 903 comprises an input port 802, an output port 804, a plurality of activation switch units 806, 808, a connection switch unit 810 and a control unit 812.

As described above, the control unit 812 is configured to, upon receiving one or more control signals indicating that an electrosurgical instrument attached to the robot arm is to be activated with a driving electrosurgical generator with a particular waveform, cause the connection switch unit 810 to be activated and, subsequent to activation of the connection switch unit 810, cause one of the activation switch units 806, 808 to be activated. The control unit 812 is configured to cause a switch unit 806, 808, 810 to be activated by causing a switch control signal for that the switch unit 806, 808, 810 to be output.

In some cases, the switch control signal output or invoked by the control unit 812 to control, or activate, a switch unit 806, 808, 810 is an A/C (alternating current) or oscillating signal. In some examples the switch control signal is a 500Hz square wave. However, it will be evident to a person of skill in the art that this is an example only. In these cases, the electrosurgical connection unit 903 may comprise a conversion circuit 902, 904, 906 per switch unit 806, 808, 810 that receives the A/C switch control signal for the corresponding switch unit 806, 808, 810 and groups the A/C pulses that form the A/C switch control signal into a single D/C (direct current) pulse, which is used to activate the switch unit 806, 808, 810. For example, the electrosurgical connection unit 903 of FIG. 9 comprises a first conversion circuit 902 that receives a first switch control signal invoked by the control unit 812 for the first activation switch unit 806 and converts that into a signal which activates the first activation switch unit 806; a second conversion circuit 904 that receives a second switch control signal invoked by the control unit 812 for the second activation switch unit 808 and converts that into a signal which activates the second activation switch unit 808; and a third conversion circuit 906 that receive a third switch control signal invoked by the control unit 812 for the connection switch unit 810 and converts that into a signal which activates the connection switch unit 810.

Each conversion circuit 902, 904, 906 may be implemented as an envelope detector. Specifically, each conversion circuit 902, 904, 906 may comprise a set of filters and diodes which are used to gradually charge a capacitor over a number of A/C pulses until the capacitor voltage switches the output of a comparator circuit. The comparator circuit may include an element of hysteresis to prevent the output changing rapidly as the capacitor charges and discharges small amounts between pulses. This means that a single pulse is incapable of causing an output signal to be output from the conversion circuit 902, 904, 906. In other words, use of an oscillating signal to activate the switch units 806, 808, 810 means that a switch unit 806, 808, 810 will not be activated if a spurious constant or momentary signal is received. Only a series of pulses in quick succession will cause an output signal to be output from a conversion circuit 902, 904, 906.

As described above, it is desirable (i) that the input port 802 and output port 804 be connected prior to sending an activation signal to the electrosurgical generator, and (ii) that the input port 802 and output port 804 be disconnected after ceasing to send an activation signal to the electrosurgical generator. This ensures that the instrument attached to the robot arm 702 is ready to receive a driving electrosurgical signal once one is output from the electrosurgical generator and continues to receive the driving electrosurgical signal throughout the entire period that it is output from the electrosurgical generator.

Accordingly, it is desirable, when an instrument is to be activated with a driving electrosurgical signal, that the appropriate activation switch unit 806, 808 be activated after the connection switch unit 810 is activated and, and in particular, after the connection switch unit has established a connection between the input port 802 and the output port 804. Specifically, there may be a delay (which may be referred to as the operation time) from when the connection switch unit 810 receives a signal indicating the connection switch unit is to be activated to when the connection switch unit 810 has established an electrical connection between the input port 802 and the output port 804, and desirably the appropriate activation switch unit 806, 808 is activated after this delay. The amount of the delay may be based on the specific components used to implement the connection switch unit 810. For example, some connection switch unit 810 implementations may have a 1 ms delay from when they receive an activate signal to when they are completely active (e.g., have established an electrical connection between the input port and the output port).

Similarly it is desirable, when an instrument is to be deactivated (after being in an active state) that the connection switch unit 810 is deactivated after the appropriate activation switch unit 806, 808 has been deactivated, and particularly after the appropriate activation switch signal has ceased sending the activation signal. Specifically, there may be a delay (which may be referred to as the release time) from when an activation switch unit 806, 8008 ceases to receive a signal indicating that it should output an activation signal to when the activation switch unit 806, 808 ceases generating an activation signal, and desirably the connection switch unit 810 is deactivated after this delay. The amount of the delay may be based on the specific components used to implement the activation switch unit 806, 808. For example, some activation switch unit 806, 808 implementations may have a delay of 0.5 ms.

In some cases, this timing of switch unit activations and deactivations may be achieved via the conversion circuits 902, 904, 906—and, in particular, by configuring the conversion circuit 906 for the connection switch unit differently relative to the conversion circuits 902, 904 for the activation switch units 806, 808. Specifically, the conversion circuits 902, 904, 906 may be configured such that if the same signal is provided to both the input of a conversion circuit 902, 904 for an activation switch unit 806, 808 and a conversion circuit 906 for the connection switch unit 810 that the connection switch unit will be activated prior to the activation switch unit, and the connection switch unit will be deactivated after deactivation of the activation switch unit. This may be implemented by selecting different parameters for one or more components of the conversion circuits 902, 904 for the activation switch units 806, 808 relative to the parameters for the components of the conversion circuit 906 for the connection switch unit 810. For example, each conversion circuit 902, 904, 906 may have a time constant which defines the delay between receiving an input signal indicating that the corresponding switch unit is to be activated to when the conversion circuit 902, 904, 906 outputs a signal (which may be referred to as an activate signal) which causes the corresponding switch unit 806, 808, 810 to be activated.

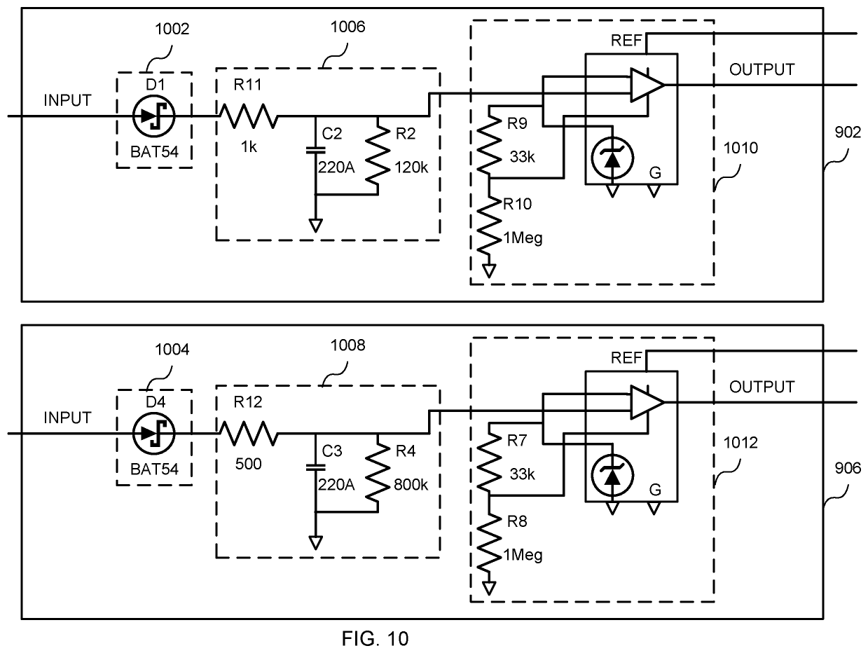

Reference is now made to FIG. 10 which illustrates an example implementation of a conversion circuit 902 for an activation switch 806 and an example implementation of a corresponding conversion circuit 906 for the connection switch unit 810 in which, when the conversion circuits 902, 906 receive the same input signal, will generate an activate at different times and cease outputting an activate signal at different times. Specifically, the conversion circuit 906 for the connection switch unit 810 will output an activate signal prior to the conversion circuit 902 for the activation switch unit 806 and will cease outputting an activate signal after the conversion circuit 902 for the activation switch unit 806.

In the example of FIG. 10, each conversion circuit 902, 906 comprises a rectifier 1002, 1004, a low pass filter 1006, 1008, and a comparator circuit 1010, 1012. As is known to those of a skill in the art, a rectifier converts an AC signal into a DC signal. Therefore, where the input to a conversion circuit 902, 906 is a pulsed AC signal the output of the rectifier 1002, 1004 is a pulsed DC signal. A rectifier 1002, 1004 can be implemented in many ways using many different components. In the example of FIG. 10 each rectifier 1002, 1004 is implemented by a Schottky diode D1, D4. However, it will be evident to a person of skill in the art that this is an example only and the rectifier may be implemented in another manner. In this example, the rectifiers 1002, 1004 of the two conversion circuits 902, 906 are implemented in the same manner.

As is known to those of skill in the art, a low pass filter is a circuit that only passes signals below its cut-off frequency while attenuating all signals above it. The low pass filters 1006, 1008 of FIG. 10 are used in conjunction with the corresponding rectifier 1002, 1004 to implement an envelope detector. In other words, together they are configured to output the envelope of the received input signal. Low pass filters generally comprise a capacitor (which is charged by the input signal), and one or more resistors (which control how quickly the capacitor is charged and discharged). For example, in FIG. 10, each low pass filter 1006, 1008 comprise a pair of resistors R11 and R2 or R12 and R4 and a capacitor C2 or C3. However, it will be evident to a person of skill in the art that is simply an example implementation of a low pass filter and that a low pass filter may be implemented in many ways using many different components. Where the input to the low pass filter is a pulsed signal, each positive pulse will charge the capacitor until the capacitor reaches a maximum charge. The capacitor will slowly discharge as it stops receiving positive pulses.

Each comparator circuit 1010, 1012 receives the output of the corresponding low pass filter 1006, 1008 and compares it to a reference voltage, and if the received signal is greater than or equal to the reference voltage it outputs a high voltage (which will activate the corresponding switch unit), and if the received signal is less than the reference voltage it outputs a low voltage (which will deactivate the corresponding switch unit). The reference voltage may be set such that it will take more than one pulse of the signal input to the conversion circuit for the capacitor to reach the reference voltage. As described above, this means that a single pulse is incapable of causing the comparator circuit, and thus the conversion circuit, from outputting a signal that will activate the corresponding switch unit. Therefore, a spurious constant or momentary signal received at the conversion circuit will not cause the corresponding switch unit to be activated. Also, since the capacitor of the low pass filter will begin discharging after a received pulse only a series of pulses in relatively quick succession will cause the corresponding switch unit to be activated. In the example of FIG. 10 each comparator circuit 1010, 1012 comprises an op-amp circuit which performs the comparison and a pair of resistors R9 and R10, or R7 and R8. However, it will be evident to a person of skill in the art that this is an example only and that a comparator circuit can be implemented in many different ways using many different components.

Since the resistors R11, R2, R12, R4 of a low pass filter 1006, 1008 control how quickly the corresponding capacitor C2, C3 charges and discharges, and thus how long after receiving a pulsed input signal it will take to charge up the capacitor C2, C3 to the reference voltage of the comparator circuit 1010, 1012 and how long it will take the capacitor C2, C3 to discharge down to the reference voltage once the pulses cease, the time between receiving a pulsed input to outputting a signal and the time between ceasing to receive a pulsed signal to ceasing to output a signal can be controlled by the resistors of the low pass filter. Accordingly, the resistors R11, R2, R12, R4 of the two low pass filters 1006, 1008 can be configured differently to cause, in response to the same input signal, the respective conversion circuits to output signals that cause the corresponding switch unit to be activated and deactivated at different times.

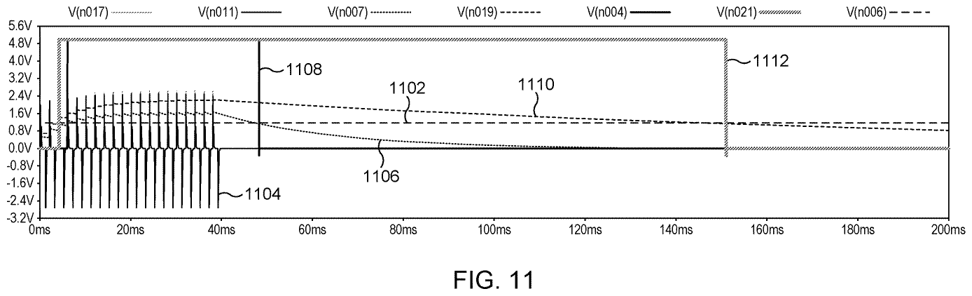

In the example of FIG. 10 the resistors R11 and R2 of the low pass filter 1006 for the activation switch unit conversion circuit 902 are set to 1 k and 120 k respectively whereas the resistors R12 and R4 of the low pass filter 1008 for the connection switch unit conversion circuit 906 are set to 500 and 800 k respectively. This causes, in response to the same input signal, the activation switch unit conversion circuit 902 to (i) output a signal that activates the activation switch unit after the connection switch unit conversion circuit 906 outputs a signal that activates the connection switch unit, and (ii) cease outputting a signal that activates the activation switch unit before the connection switch unit conversion circuit 906 ceases outputting a signal that activates the connection switch unit. This is illustrated in FIG. 11.

Specifically, FIG. 11 is a graph showing the output of the low pass filters 1006, 1008 and comparator circuits 1010, 1012 of the conversion circuits 902, 906 of FIG. 11 with a reference voltage 1102 in response to a pulsed or oscillating input signal 1104. Specifically, the outputs of the low pass filter 1006 and the comparator circuit 1010 of the activation switch unit conversion circuit 902 are shown at 1106 and 1108 of FIG. 11 respectively, and the outputs of the low pass filter 1008 and the comparator circuit 1012 of the connection switch unit conversion circuit 906 are shown at 1110 and 1112 respectively. It can be seen in FIG. 11 that, in response to the same input signal 1104: (i) the output of the low pass filter 1008 of the connection switch unit conversion circuit 906 reaches the reference voltage 1102 faster than the output of the low pass filter 1006 of the activation switch unit conversion circuit 902, thus the comparator circuit 1012 of the connection switch unit conversion circuit 906 outputs an activate signal sooner than the comparator circuit 1010 of the activation switch unit conversion circuit 902; and (ii) the output of the low pass filter 1008 of the connection switch unit conversion circuit 906 falls to the reference voltage 1102 slower than the output of the low pass filter 1006 of the activation switch unit conversion circuit 902, thus the comparator circuit 1012 of the connection switch unit conversion circuit 906 ceases outputting an activating signal after the comparator circuit 1010 of the activation switch unit conversion circuit 902 ceases outputting an activate signal. Accordingly, if the two conversion circuits 902, 906 are sent the same signal at the same time this will cause the connection switch unit to be activated prior to the respective activation switch unit and the connection switch unit to be deactivated after the respective activation switch unit.

Returning to FIG. 9, it may be advantageous to isolate the control unit 812 from the input active wire 814 so that the high-powered driving electrosurgical signal carried thereon does not cause damage to the one or more processors 842, memory 844 and/or communications module 840 thereof. Specifically, it may be beneficial to pass any (direct or indirect) electrical path between the control unit 812 and the input active wire 814 through an isolation barrier. The switch units 806, 808, 810 themselves provide one isolation barrier for the control unit 812. However, in some cases this may not be sufficient to ensure that the control unit 812 is protected from the high power driving electrosurgical signals.

Accordingly, in some cases, the electrosurgical connection unit 903 may also comprise an isolation device 908 (which may be referred to herein as the main isolation device) that establishes an isolation barrier between the control unit 812 and the input active wire 814. In these cases, any electrical path between the control unit 812 and the input active wire 814, such as the paths used to transmit switch control signals to the switch units 806, 808, 810, is established via the isolation device 908 so that control signals are provided to the corresponding switch unit 806, 808, 810 in a manner that ensures that any high powered signal transmitted or carried on that path is not transmitted to the control unit 812. The isolation device 908 may be any suitable isolation device such as a digital isolator or an opto-isolator. As is known to those of skill in the art, digital isolators use semiconductor process technology to create either transformers or capacitors to transfer electrical signals between two isolated circuits, whereas opto-isolators transfer electrical signals between two isolated circuits using light.

Since a failure of the control unit 812 or the isolation device 908 is likely to result in an erroneous D/C signal (rather than an A/C signal) the electrosurgical connection unit 903 may comprise one or more A/C coupling circuits 910, 912, 914 that precede one or more of the conversion circuits 902, 904, 906 to ensure that a failure of the control unit 812 or the isolation device 908 cannot lead to inadvertent activation of a switch unit 806, 808, 810. Each A/C coupling circuit 910, 912, 914 is configured to receive a signal and filter out the D/C (direct current) component of the signal and output only the A/C component of the signal. Each A/C coupling circuit 910, 912, 914 may comprise one or more capacitors. In some examples there may be an A/C coupling circuit 910, 912, 914 situated between the control unit 812 (or the isolation device 908 if there is one) and each conversion circuit 902, 904, 906 that is configured to receive the corresponding switch control signal from the control unit 812 (or the isolation device 908 if there is one) and A/C couple this signal to the conversion circuit 902, 904, 906 so that the conversion circuit 902, 904, 906 receives an AC only signal (and any D/C component, erroneous or otherwise is removed). For example, the electrosurgical connection unit 903 shown in FIG. 9 comprises a first A/C coupling circuit 910 situated between the isolation device 908 and the first conversion circuit 902 which is configured to receive a switch control signal invoked by the control unit 812 and output the A/C component of that signal; a second A/C coupling circuit 912 situated between the isolation device 908 and the second conversion circuit 904 which is configured to receive a switch control signal invoked by the control unit 812 and output the A/C component of that signal; and a third A/C coupling circuit 914 that is situated between the isolation device 908 and the third conversion circuit 906 which is configured to receive a switch control signal invoked by the control unit 812 and output the A/C component of that signal.

As described above with respect to FIG. 8, it could be quite dangerous if a switch unit 806, 808, 810 of the electrosurgical connection unit 903 failed such that it was stuck in an activated state (i.e. the switches 834, 836, 838 of the switch unit 806, 808, 810 are stuck in a closed position) because this could allow an activation signal to be inadvertently transmitted to the electrosurgical generator and/or the electrosurgical generator to be inadvertently connected to an electrosurgical instrument. As a result, the electrosurgical connection unit may comprise one or more measurement units 916, 918 that are configured to measure a parameter of one or more switch units 806, 808, 810 and transmit measurement information to the control unit 812 which can be used to determine whether the switch unit 806, 808, 810 is working properly.

In some examples, each measurement unit 916, 918 may be an impedance measurement unit configured to measure the impedance across a switch unit 806, 808, 810. In these cases, an impedance measurement unit 916, 918 for an activation switch unit may be electrically coupled to both the input active wire 814 and the control wire 816, 818 of the relevant activation switch unit 806, 808 to measure the impedance between them. As is known to those of skill in the art, the impedance between two points of a circuit may be determined, for example, by applying a current or voltage at one point and measuring the current or voltage at the other point. However, in other examples, the measurement unit(s) may be configured to measure another parameter of the switch units 806, 808, 810 such as voltage or current.

In some cases, the one or more processors 842 may be configured to control the operation of the measurement units 916, 918. For example, the one or more processors 842 may be configured to periodically place a switch unit 806, 808, 810 in a deactivated state (i.e. a state in which the switches of the switch unit are in the open position) when the electrosurgical generator is inactive (i.e. is not outputting a driving electrosurgical signal) and then cause the measurement unit to measure the desired parameter (e.g. impedance). In these cases, when the system is started up, an initialisation test may be performed to determine a benchmark measurement for the parameter (e.g. impedance) when the switch unit 806, 808, 810 is in the deactivated state. This benchmark can then be compared against the measured parameter to determine if any of the switches in that switch unit is erroneously in the closed position.

In addition, or alternatively, the one or more processors may be configured to periodically place a switch unit 806, 808, 810 in an activated state (i.e. a state in which the switches of the switch unit are in the closed position) when the electrosurgical generator is inactive (i.e. not outputting a driving electrosurgical signal) and then cause the measurement unit 916, 918 to measure the desired parameter (e.g. impedance). This measurement can be used to determine if the electrosurgical generator is active when an activation signal has not been transmitted to the electrosurgical generator.

In the example shown in FIG. 9 there is a measurement unit 916, 918 for each activation switch unit 806, 808. However this is an example only and in other examples there may be more or fewer measurement units. For example, the electrosurgical connection unit may only comprise a measurement unit for one activation switch unit 806, 808, and/or the electrosurgical connection unit may also, or alternatively, comprise a measurement unit for the connection switch unit 810 which operates in the same manner as the measurement units 916, 918 for the activation switch units 806, 808.

In FIG. 9 the electrosurgical connection unit 903 comprises a first measurement unit 916 that is configured to measure a parameter (e.g. impedance) of the first activation switch unit 806 and a second measurement unit 918 that is configured to measure a parameter (e.g. impedance) of the second activation switch unit 808. In other cases, such as where the activation switch units 806, 808 comprise two or more switches in series, there may be one measurement unit 916, 918 per switch. For example, where each activation switch unit 806, 808 comprises two switches in series, the electrosurgical connection unit 903 may comprise four measurement units—a first measurement unit that measures a parameter (e.g. impedance) across the first switch of the first activation switch unit 806, a second measurement unit that measures a parameter (e.g. impedance) across the second switch of the first activation switch unit 806, a third measurement unit that measures a parameter (e.g. impedance) across the first switch of the second activation switch unit 808, and a fourth measurement unit that measures a parameter (e.g. impedance) across the second switch of the second activation switch unit 808. However, in other cases there may be a single measurement unit that is configured to measure the parameter of multiple activation switch units 806, 808.

In some cases, the one or more processors 842 may be configured to receive the measurement information (e.g. the value of the measured parameter) from a measurement unit 916, 918 and analyse the received measurement information to determine whether the measurement information indicates that one or more of the switch units 806, 808, 810 is/are not operating as expected and/or the electrosurgical generator is not operating as expected. For example, where the measurement unit is an impedance measurement unit, the one or more processors 842 may be configured to determine that one or more of the switch units 806, 808, 810 is not operating as expected if a switch unit is expected to be in a deactivated state (i.e. the switches thereof are in an open position) yet there is no (or very little) impedance across the switch unit 806, 808. In response to determining that at least one of the switch units 806, 808, 810 is not operating as expected or the electrosurgical generator is not operating as expected the one or more processors 842 may be configured to send an error notification to the external computing device (e.g. robot control unit 718) via the communications module 840.

In other cases, the one or more processors 842 may be configured to simply receive the measurement information from the measurement unit(s) 916, 918 and transmit the measurement information to the external computing device (or another computing device), via the communications module 840, for further analysis and processing.

Where one or more measurement units 916, 918 transmit measurement information to the control unit 812, the electrical path between the control unit 812 and the measurement unit 916, 918 may be established via the isolation device 908 so that any high powered signal that passes through the measurement unit 916, 918 is not transmitted to the control unit 812.

In some cases, the isolation device 908 may not provide sufficient protection for the control unit 812 from the high powered signals that may pass through a measurement unit 916, 918. Accordingly, the electrosurgical connection unit 903 may further comprise one or more additional isolation devices 920, 922 between the measurement units 916, 918 and the control unit 812 to provide double isolation between the measurement unit and the control unit 812, like the double isolation that is provided between the switch units 806, 808, 810 via the main isolation device 908 and the switches of the switch units. Specifically, in some cases, there may be an additional isolation device 920, 922 that is situated between each measurement unit 916, 918 and the isolation device 908. For example, in the electrosurgical connection unit 903 of FIG. 9 there is a first additional isolation device 920 that is situated between the first measurement unit 916 and the main isolation device 908, and a second additional isolation device 922 that is situated between the second measurement unit 918 and the main isolation device 908. In some cases, one or more of the additional isolation devices 920, 922 may be an opto-isolator, which may also be referred to as an optocoupler, photocoupler, or optical isolator. As is known to those of skill in the art, an opto-isolator, in contrast to a digital isolator, transfers electrical signals between two isolated circuits using light.

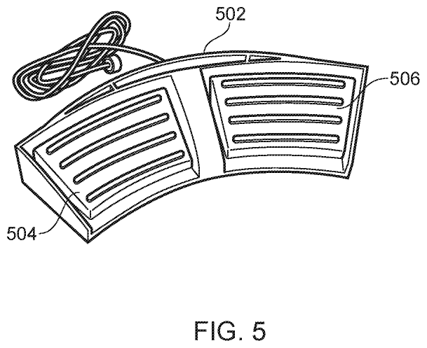

In some cases, the control logic 728 of the electrosurgical generator 726 may be configured to detect an activation signal on a control line by measuring the impedance on the line. The control logic 728 may also be able to detect a fault or failure based on the measured impedance. In existing manual, as opposed to, robotic electrosurgical systems, such as those described above with respect to FIGS. 3-6, wherein an electrosurgical generator 726 is controlled by controls on an electrosurgical instrument or a foot pedal system, the wires in the cables connecting the electrosurgical generator to the electrosurgical instrument (FIGS. 3-4), or foot pedal system (FIGS. 5-6) typically present a specific capacitance to the electrosurgical generator 726 and the control logic 728 is configured to detect an activation signal and identify a fault condition based on that amount of capacitance on the line. In the robotic electrosurgical systems described herein wherein the electrosurgical generator 726 is controlled by an electrosurgical connection unit the wires in the cables connecting the electrosurgical generator to the electrosurgical connection unit may present a different amount of capacitance to the electrosurgical generator 726 compared to the wires in the cables used in manual electrosurgical systems. In some examples, they may present less capacitance and in other examples, they may present more capacitance. For example, in some cases, the wires in the cables used in the robotic electrosurgical systems described herein may be shorter than the wires in the cables used in manual electrosurgical systems, and thus have less capacitance than the wires in the cables used in manual electrosurgical systems. In yet other cases, the wires in the cables use din the robotic electrosurgical systems describe herein may have more capacitance than the wires in the cables used in manual electrosurgical systems.

In these cases, to ensure that the electrosurgical generator 726 can correctly detect activation signals and to prevent the electrosurgical generator 726 from erroneously detecting a fault condition on the control line, the electrosurgical connection unit 903 may comprise one or more capacitance emulation units 924, 926 wherein each capacitance emulation unit 924, 926 is connected across one of the control wires 816, 818 and the input active wire 814. Each capacitance emulation unit 924, 926 comprises one or more capacitors 928, 930 and/or one or more other capacitive components that are configured to add or subtract capacitance in an effort to emulate the capacitance of the corresponding wire in the cables used in manual electrosurgical systems. For example, in FIG. 9, there is a first capacitance emulation unit 924 that comprises a single capacitor 928 across the first control wire 816 and the input active wire 814; and a second capacitance emulation unit 926 that comprises a single capacitor 930 across the second control wire 818 and the input active wire 814. The total capacitance presented by each capacitance emulation unit 924, 926 may be based on the difference between the capacitance presented by the wires in the cables used to connect the electrosurgical connection unit 903 to the electrosurgical generator 726 and the capacitance expected by the electrosurgical generator 726. It will be evident to a person of skill in the art that this is an example only and that the capacitance emulation units 924, 926 may take any suitable form that allows them to add or subtract capacitance from a control line.

It will be evident to a person of skill in the art that FIG. 9 illustrates an example set of components that may form part of an electrosurgical connection unit and an electrosurgical connection unit may comprise any combination of: one or more conversion circuits 902, 904, 906; a main isolation device 908; one or more A/C coupling circuits 910, 912, 914; one or more measurement units 916, 918; one or more additional isolation devices 920, 922; and one or more capacitance emulation units 924, 926.

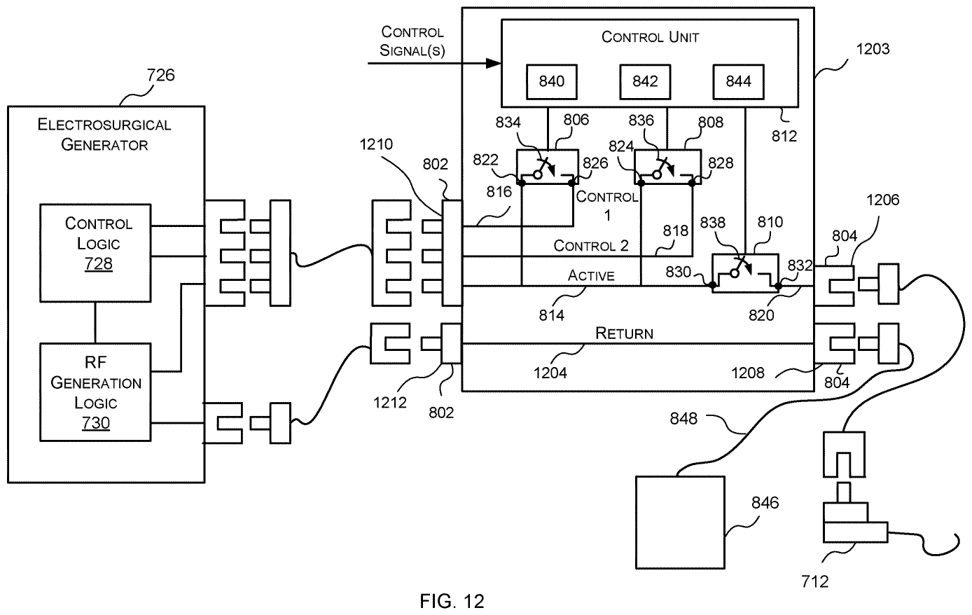

Reference is now made to FIG. 12 which illustrates a third example electrosurgical connection unit 1203 for connecting a monopolar electrosurgical instrument to an electrosurgical generator 726. The example electrosurgical connection unit 1203 of FIG. 12 is the same as the electrosurgical connection unit 703 of FIG. 8 except that instead of the return electrode 846 being directly connected to the electrosurgical generator 726, the return electrode is connected to the electrosurgical connection unit 1203 and the return electrosurgical signal received from the return electrode 846 is transmitted to the electrosurgical generator via the electrosurgical connection unit 1203. In this example, the output port 804 is configured to receive the return electrosurgical signal from the return electrode 846 and transmit the received return electrosurgical signal on a return wire 1204. The return wire 1204 is also coupled to the input port 802 to allow any received return electrosurgical signal to be output on the input port 802.

In the example shown in FIG. 12, the output port 804 of the electrosurgical connection unit 1203 comprises a first connector 1206 that is configured to engage a corresponding connector connected to a cable that is connected to the electrosurgical instrument 712, and a second connector 1208 that is configured to engage a corresponding connector connected to a cable 848 that is connected (directly or indirectly) to the return electrode 846. In other examples, the output port 804 may comprise a single connector that is configured to engage a corresponding connector that is connected to two cables—one of which is connected (directly or indirectly) to the electrosurgical instrument 712, and the other of which is connected (directly or indirectly) to the return electrode 846. The output port 804 connector(s) may be female, and the corresponding connectors may be male or vice versa. In many cases, the input port 802 and the output port 804 have opposite connectors to avoid electrosurgical devices from being plugged into or connected to the wrong port (i.e. to avoid an electrosurgical instrument being inadvertently plugged into the input port 802 and/or an electrosurgical generator 726 being inadvertently plugged into the output port 804). For example, in some cases the input port 802 may have male connectors(s) and the output port 804 may have female connector(s).

In the example shown in FIG. 12, the input port 802 of the electrosurgical connection unit 1203 comprises a first connector 1210 that is configured to engage a corresponding connector connected to a cable that is connected (directly or indirectly) to the electrosurgical generator 726 and is configured to carry the driving electrosurgical signal and control signals between the electrosurgical generator 726 and the electrosurgical connection unit 1203; and a second connector 1212 that is configured to engage a corresponding connector connected to a cable that is connected (directly or indirectly) to the electrosurgical generator 726 and is configured to carry the return electrosurgical signal from the electrosurgical connection unit 1203 and the electrosurgical generator 726. In other examples, the input port 802 may comprise a single connector that is configured to engage a corresponding connector that is connected to a cable connected (directly or indirectly) to the electrosurgical generator 726. In yet other examples, the input port 802 may have any number of connectors that are configured to engage corresponding connectors to enable the driving electrosurgical signal, the control signals and the return electrosurgical signal to be transmitted between the electrosurgical generator 726 and the electrosurgical connection unit 1203. The input port 802 connector(s) may be male and the corresponding connectors which engage the input port 802 connectors may be female or vice versa.

In the electrosurgical connection unit 1203 of FIG. 12, the electrical connection between the return electrode 846 and the electrosurgical generator 726 is established via the electrosurgical connection unit 1203 of FIG. 12 thus there is a second electrical or current path between the electrosurgical generator 726 and the surgical site that is established via the electrosurgical connection unit 1203. However, the electrosurgical connection unit 1203 does not comprise a second connection switch unit that is used to break and establish this second electrical or current path in the same manner as the connection switch unit 810 is used to break and establish the electrical or current path between the electrosurgical generator 726 and the electrosurgical instrument 712. This is because it is unlikely that the current applied to another electrosurgical instrument at the same surgical site as the monopolar electrosurgical instrument 712 of FIG. 12 will return via this second electrical or current path and, even if it does, it is unlikely to cause injury (e.g. burns) to the patient due to the large surface area of a typical return electrode 826, which is generally in the form of a pad. Specifically, if the other electrosurgical instrument is a bipolar electrosurgical instrument, the voltage of the electrosurgical activation signals for bipolar electrosurgical instruments tends to be lower than the voltage of electrosurgical activation signals for monopolar electrosurgical instruments, such as the electrosurgical instrument 712 of FIG. 12, making it less likely that the energy or current will return via the return electrode 846; and if the other electrosurgical instrument is a monopolar electrosurgical instrument the two monopolar instruments are likely to share a single return electrode 848 thus it would not be desirable to disconnect the return electrode 846 from the electrosurgical generator even when the electrosurgical instrument 712 is not in use. However, in other examples, the electrosurgical connection unit 1203 may further comprise a second connection switch unit that operates in the same manner as the connection switch unit 810 to dynamically break and establish the electrical connection (or electrical or current path) between the return electrode 846 and the electrosurgical generator 726.

Reference is now made to FIG. 13 which illustrates an example electrosurgical connection unit 1303 for connecting a bipolar electrosurgical instrument 1304 to an electrosurgical generator 726. As described above, a bipolar electrosurgical instrument comprises both an active electrode 1306 and a return electrode 1308. The active electrode 1306 is activated by a driving electrosurgical signal generated by the electrosurgical generator 726 and the return electrode 1308 receives the return electrosurgical signal which is transmitted to the electrosurgical generator 726. The active and return electrodes 1306, 1308 may be made of, or may comprise, an electrically conductive type of material, such as, for example, stainless steel.

The electrosurgical connection unit 1303 of FIG. 13 is the same as the electrosurgical connection unit 1203 of FIG. 12 in that the return electrosurgical signal is received on the output port 804 and transmitted out the input port 802. However the electrosurgical connection unit 1303 has only one activation switch unit 808 (vs the two activation switch units 806, 808 in the electrosurgical connection unit 1203 of FIG. 12), the return wire is divided into an input return wire 1310 coupled to the input port 802 and an output return wire 1312 coupled to the output port 804, and there is a second connection switch unit 1314 situated between the input and output return wires 1310, 1312.

There is only one activation switch unit 808 because many bipolar instruments can only be driven by a driving electrosurgical signal with one type of waveform-a bipolar waveform. However, it will be evident to a person of skill in the art that this is an example only and that other bipolar instruments may be able to support driving electrosurgical signals with multiple different waveforms. Since there is only one activation switch unit 808 in this example, there is also only one control wire 818. When the activation switch unit 808 is activated it sends an activation signal to the electrosurgical generator 726 that indicates that a driving electrosurgical signal with a bipolar waveform is to be activated which, when detected by the electrosurgical generator 726 causes the electrosurgical generator 726 to output a driving electrosurgical signal with a bipolar waveform.

The electrosurgical connection unit 1303 of FIG. 13 has a second connection switch unit 1314, because a bipolar instrument has two electrical paths to the electrosurgical generator 726—the active path and the return path—and to reduce the chance of the current from another instrument taking one of those paths, both paths to the electrosurgical generator 726 are disabled or disconnected when the electrosurgical instrument is not in use, Thus the second connection switch unit 1314 disconnects the return path between the bipolar electrosurgical instrument 1304 and the electrosurgical generator when the bipolar electrosurgical instrument 1304 is not in use. Specifically, the second connection switch unit 1314 is configured to, when activated, cause the input port 802 to be electrically connected to the output port 804 such that any return electrosurgical signal received on the output port 804 is output on the input port 802.

In the example shown in FIG. 13, a first port 1316 of the second connection switch unit 1314 is connected to the input return wire 1310 and a second port 1318 of the second connection switch unit 1314 is connected to the output return wire 1312. In this example, when the second connection switch unit 1314 is activated, the input return wire 1310 is electrically connected to the output return wire 1312 (i.e. the input return wire 1310 is shorted to the output return wire 1312) which causes a return electrosurgical signal received on the output port 804 to be output on the input port 802. Like the first connection switch unit 810, the second connection switch unit 1314 may comprise one or more switches 1320.

It is desirable that both connection switch units 810, 1314 be activated and deactivated at the same time so that the entire instrument is either connected or disconnected from the electrosurgical generator. Accordingly, the control unit 812 may be configured to, in response to receiving a control signal (or a set of control signals) indicating that the electrosurgical instrument connected to the arm is to be activated, generate or invoke switch control signals that cause both connection switch units to be activated at the same time.

The output port 804 may comprise multiple connectors. For example, the output port 804 may have one connector which is configured to engage a corresponding connector connected to a cable that is configured to carry the driving electrosurgical signal from the electrosurgical connection unit 1303 to the bipolar electrosurgical instrument 1304, and a second connector which is configured to engage a corresponding connector connected to a cable that is configured to carry the return electrosurgical signal from the bipolar electrosurgical instrument 1304 to the electrosurgical connection unit 1303. However, in other cases the output port 804 may comprise a single connector as shown in FIG. 13 that is configured to engage a corresponding connector which is connected to a cable that is configured to carry both the driving electrosurgical signal to, and the return electrosurgical signal from, the bipolar electrosurgical instrument 1304.

Although the electrosurgical connection units 703, 903, 1203 and 1303 of FIGS. 8, 9, 12 and 13 were described as supporting either a bipolar electrosurgical instrument or a monopolar electrosurgical instrument, other example electrosurgical connection units may comprise components to support both monopolar electrosurgical instruments and bipolar electrosurgical instruments. Such electrosurgical connection units may comprise all the components of the electrosurgical connection unit 803, 903, or 1203 described above to support a monopolar electrosurgical instrument and all of the components of the electrosurgical connection unit 1303 of FIG. 13 to support a bipolar electrosurgical instrument. For efficiency such electrosurgical connection units may comprise a single control unit that controls all of the switch units (i.e. the activation switch units and connection switch unit that control activation of a monopolar electrosurgical instrument; and the activation switch unit and the control switch units the control activation of a bipolar electrosurgical instrument). An electrosurgical instrument attached to the arm may then be dynamically connected to the monopolar components or the bipolar components depending on whether the electrosurgical instrument is a monopolar electrosurgical instrument or a bipolar electrosurgical instrument.

Any of the electrosurgical connection units 703, 903, 1203, or 1303 described above with respect to FIGS. 8, 9, 12, and 13 may comprise any combination of the optional features described above with respect to FIG. 9. Specifically, any of the electrosurgical connection units described herein may comprise any combination of: one or more conversion circuits 902, 904, 906; a main isolation device 908; one or more A/C coupling circuits 910, 912, 914; one or more measurement units 916, 918; one or more additional isolation devices 920, 922; and one or more capacitance emulation units 924, 926.

Reference is now made to FIG. 14 which illustrates an example method 1400 which may be executed by the control unit 812 (e.g. the one or more processors 842 of the control unit) of the electrosurgical connection unit 703, 903, 1203, 1303 to cause (i) an activation signal to be output on the input port 802 of the electrosurgical connection unit, and (ii) the input port 802 be connected to the output port 804 such that any driving electrosurgical signal received on the input port 802 in response to the activation signal is output on the output port 804. As described above, the electrosurgical connection unit 703, 903, 1203, 1303 comprises a control unit, one or more activation switch units and a connection switch unit.

The method 1400 begins at block 1402 where the control unit 812 (e.g. the one or more processors 842 of the control unit 812) of the electrosurgical connection unit determines whether it has received (e.g. via the communications module 840) a control signal or a set of control signals from an external computing device. If the control unit 812 (e.g. the one or more processors 842) determines that the control unit 812 has received a control signal, or a set of control signals, then the method 1400 proceeds to block 1404. If, however, the control unit 12 (e.g. the one or more processors 842) determines that the control unit 812 has not received a control signal or a set of control signals then the method 1400 proceeds back to block 1402.

At block 1404, the control unit 812 (e.g. the one or more processors 842 thereof) determines whether the control signal or set of controls signals indicate that an electrosurgical instrument attached to the arm is to be activated by a driving electrosurgical signal. If the control unit 812 (e.g. the one or more processors 842 thereof) determines that the control signal or set of control signals indicate that an electrosurgical instrument attached to the arm is to be activated, then the method 1400 may proceed to block 1406 or block 1410. Whether the method 1400 proceeds to block 1406 or 1410 may depend on whether the electrosurgical connection unit has more than one activation switch unit or supports more than one type of driving electrosurgical signal. For example, if the electrosurgical connection unit comprises only one activation switch unit, then the method 1400 may proceed directly to block 1410 and if the electrosurgical connection unit comprises multiple activation switch units, then the method 1400 may proceed to blocks 1406 and 1408 where a determination is made as to which activation switch unit is to be activated. If, however, the control unit 812 (e.g. the one or more processors 842 thereof) determines that the control signal or set of control signals do not indicate that the electrosurgical instrument attached to the arm is to be activated then the method 1400 proceeds back to block 1402.

At block 1406, the control unit 812 (e.g. the one or more processors 842) determines from the control signal, or set of control signals, the waveform to be used for the driving electrosurgical signal. Specifically, the electrosurgical generator may comprise multiple activation switch units each of which, when activated, notify the electrosurgical generator that the electrosurgical generator should generate a driving electrosurgical signal with a particular waveform. For example, as described above, an electrosurgical connection unit may have a first activation switch unit, which when activated, causes a first activation signal to be output from the input port of the electrosurgical connection unit which indicates to an electrosurgical generator that a driving electrosurgical signal with a first waveform is to be generated; and a second activation switch unit, which when activated, causes a second activation signal to be output from the input port of the electrosurgical connection unit which indicates to an electrosurgical generator that a driving electrosurgical signal with a second waveform is to be generated. Accordingly, when the electrosurgical connection unit comprises multiple activation switch units, the control unit 812 (e.g. the one or more processors 842) may analyse the control signal, or set of control signals, to determine which waveform is to be used for the driving electrosurgical signal. Once the control unit 812 (e.g. the one or more processors 842) has determined the desired waveform for the driving electrosurgical signal the method 1400 proceeds to block 1408.

At block 1408, the control unit 812 (e.g. one or more processors 842 thereof) determines, from the desired waveform identified in block 1406, the appropriate activation switch unit of the plurality of activation switch units to be activated to cause an activation signal identifying the desired waveform to be output. For example, if the desired waveform is a COAG waveform, then the control unit 813 (e.g. one or more processors 842 thereof) may identify the activation switch unit 806, 808 associated with the COAG waveform. Where the electrosurgical connection unit also comprises components for both bipolar and monopolar electrosurgical instruments then the electrosurgical connection unit may also comprise multiple connection switch units. In such cases, the desired waveform may also indicate which connection switch unit(s) is/are to be activated (e.g. whether the monopolar or bipolar connection switch unit(s) are to be activated). Once the appropriate activation switch unit (and optionally, the appropriate connection switch unit(s) has/have been identified, the method 1400 proceeds to block 1410.

At block 1410, the control unit 812 (e.g. one or more processors 842 thereof) generates or invokes one or more signals that cause (i) the (appropriate) connection switch unit 810 to be activated so that the input port 802 is electrically connected to the output port 804 such that a driving electrosurgical signal received on the input port 802 will be output on the output port 804; and (ii) the appropriate activation switch unit 806, 808 to be activated, subsequent to the connection switch unit 810, so that an activation signal is output from the input port 802. As described above, the timing of the switch unit activations may be implemented in software or hardware. For example, in some cases the control unit 812 may be configured to, in response to receiving a control signal indicating that the electrosurgical instrument attached to the arm is to be activated or energised, generate or invoke a first switch control signal indicating that the connection switch unit is to be activated and subsequently generate or invoke a second switch control signal indicating that the appropriate activation switch unit is to be activated.

However, in other examples, the electrosurgical connection unit 703, 903, 1203, 1303 may comprise a hardware unit or circuit (e.g. conversion circuit 902, 904, 906) for each switch unit that is situated between the control unit and that switch unit. Each such hardware unit or circuit is configured to receive the switch control signal generated, or invoked, by the control unit 812 for the corresponding switch unit and activate (and deactivate) the switch unit at the correct time. In particular, if the hardware unit for the connection switch unit and the hardware unit for the activation switch unit receive the same switch control signal, the hardware unit for the connection switch unit will activate the connection switch unit prior to the hardware unit for the activation switch unit activating the activation switch unit, and the hardware unit for the connection switch unit will deactivate the connection switch unit after the hardware unit for the activation switch unit deactivates the activation switch unit. In these cases, the control unit 812 may be configured, in response to receiving a control signal indicating that the electrosurgical instrument attached to the robot arm is to be activated, invoke or generate, at the same time, a first control signal indicating that the connection switch unit is to be activated and a second switch control signal indicating that the appropriate activation switch unit is to be activated—and then the hardware units or circuits will take care of the timing of the activations (and deactivations) of the connection switch unit and the activation switch unit. Implementing the timing of the activation and deactivation of the connection switch unit and the appropriate activation switch unit in hardware, vs software, may be safer (e.g. less prone to error).

In some cases, the control unit 812 may be configured to output one or more switch control signals that cause the appropriate switch units (activation switch unit and connection switch unit) to be activated for only a predetermined period (e.g. a few milliseconds) and then the method 1400 proceeds back to block 1402 where the control unit 812 determines whether it has received a new control signal (or set of control signals) indicating that the instrument attached to the robot arm should continue to be activated with a driving electrosurgical signal. In this way the control unit 812 only causes the appropriate switch units 806, 808, 810 to be activated while the control unit 812 continues to receive a control signal (or a set of control signals) indicating that the instrument attached to the associated arm is to be activated with a driving electrosurgical signal. The predetermined period may be quite short (e.g. a few milliseconds) to allow the control unit 812 to respond quickly to a change from activation to deactivation (or vice versa) and from one type of activation to another (e.g. from a driving electrosurgical signal with a first waveform to a driving electrosurgical signal with a second waveform).

In some cases, the control unit 812 may be configured to implement the method 1400 of FIG. 14 using a token-based approach to verify the latency between the control unit 812 and the external computing device (e.g. robot control unit 718) generating the control signal(s) so that stale control signals can be ignored. In the token-based approach the control unit 812 is configured to generate a new token on a periodic basis. For example, the control unit 812 may be configured to generate a new token at a frequency of 1 kHz. The token comprises information that indicates the time at which the token was generated. For example, the control unit 812 may be configured to update a rolling counter (e.g. a 16-bit counter) each period and include the latest counter value in the token for that period. The token may also comprise information that uniquely identifies the electrosurgical connection unit to which the control unit 812 belongs (e.g. an electrosurgical connection unit identifier (ID)).

In some cases, the token may also comprise validation information which indicates whether the token is valid (e.g. has not been corrupted). For example, the token may also comprise a CRC (cyclic redundancy check) value based on some or all the information (e.g. fields) in the token. In some cases, each token may comprise an 8-bit CRC value.

Once the token has been generated, the control unit 812 transmits, directly or indirectly, (e.g. via the communication module 840) the generated token to the external computing device (e.g. robot control unit 718) that generates the control signal for the electrosurgical connection unit.

The external computing device (e.g. robot control unit 718) receives the token and, if the external computing device receives information indicating that the electrosurgical instrument attached to the electrosurgical connection unit identified in the token is to be activated by a driving electrosurgical signal with a particular waveform, the token is modified to indicate the particular waveform to be generated and the updated token is transmitted back to the control unit 812. For example, as described above, the command interface may comprise a display and one or more hand controllers or joysticks. The surgeon, or other user, may be able to select, via a graphical user interface displayed on the display, the waveform to be generated and the electrosurgical instrument to be activated. The surgeon, or other user, may then be able to indicate that the selected electrosurgical instrument is to be activated with the selected waveform by pressing an electrosurgical activation button on the hand controller or joystick. In these examples, when the user presses the electrosurgical activation button, the most recent token related to the electrosurgical connection unit to which the selected arm is attached is updated with information indicating the selected waveform. Where the token includes validation information the validation information (e.g. CRC value) may be updated to reflect the waveform information added to the token.

Where, for example, the electrosurgical generator supports three different waveforms (e.g. a monopolar COAG waveform, a monopolar CUT waveform, and a bipolar waveform) the token may comprise a two-bit waveform field which indicates the selected waveform. For example, a “01” (binary) in the waveform field may indicate a monopolar COAG waveform, a “10” (binary) in the waveform field may indicate a monopolar CUT waveform, and a “11” (binary) may indicate a bipolar waveform. It will be evident to a person of skill in the art that this is an example only and in other examples the waveform field may have more, or fewer bits based on the number of different waveforms supported by the electrosurgical generators used to drive the electrosurgical instrument(s). Specifically, to support more waveforms the waveform field may comprise more bits. Examples of additional waveforms that may be supported include, but are not limited to, a BLEND waveform (described above).

In some cases, the external computing device (e.g. robot control unit 718), or one or more other devices which receive the token or the modified token prior to the electrosurgical connection unit, may be configured to negate the token or the modified token if one or more conditions for activating the selected electrosurgical instrument with the selected waveform are not met (or, alternatively, when a fault condition is detected). For example, the external computing device (e.g. robot control unit 718) and/or one or more other devices may be configured to negate a token or a modified token if and when any of the following conditions are detected: (i) the user is not currently controlling an arm that is connected to an electrosurgical instrument (e.g. the hand controller or joystick on which the activation button was pressed is not actively connected to an arm that is connected to an electrosurgical instrument); (ii) the validation information indicates the token is invalid (e.g. a CRC check fails); (iii) the electrosurgical instrument attached to the selected arm does not support the selected waveform; (iv) the electrosurgical instrument, the arm or the electrosurgical connection unit is in a fault mode; and (v) the communications network (e.g. Ethernet network) over which the external computing device and the electrosurgical connection unit communicate is faulty. It will be evident to a person of skill in the art that these are examples only and that other conditions or fault states may cause a token or a modified token to be negated. In some cases, negating the token or modified token may comprise setting all fields of the token or modified token (including the validation field (where there is a validation field)) to zero which may be referred to as a zeroed token. In some cases, a negated token or negated modified token is not passed on to the electrosurgical connection unit.

When the modified token is received at the electrosurgical connection unit the electrosurgical connection unit is deemed to have received a control signal (block 1402 of method 1400). The control unit 812 then determines whether the modified token indicates that an electrosurgical instrument attached to the electrosurgical connection unit is to be activated (block 1404 of method 1400). The control unit 812 may determine that the modified token indicates that an electrosurgical instrument attached to the electrosurgical connection unit is to be activated (i) if the information in the token identifying the electrosurgical connection unit that generated the token matches the identifying information for the current electrosurgical connection unit; and (ii) if the information indicating when the token was generated (counter information) indicates that less than a predetermined amount of time has elapsed since the token was generated. In some cases, the control unit 812 may determine that less than a predetermined amount of time has elapsed since the token was generated by comparing the counter information in the token to the current value of the counter (e.g. by computing the difference) and determining whether the difference exceeds a threshold. In some cases, the threshold may be set so that the predetermined time is only a few milliseconds.

Where the modified token comprises validation information (e.g. a CRC value) the control unit 812 may only determine that the token indicates that an electrosurgical instrument attached to the electrosurgical connection unit is to be activated if the above conditions are met and the validation information indicates that the token is valid (e.g. a CRC check passes).

If the control unit 812 determines that the modified token indicates that an electrosurgical instrument attached to the electrosurgical connection unit is to be activated, then the control unit 812 may analyse the modified token to identify the desired waveform for the driving electrosurgical signal (block 1406 of method 1400).

Once the control unit 812 identifies the desired waveform for the driving electrosurgical signal the control unit 812 identifies the activation switch unit 806, 808 associated with the desired waveform (block 1408 of method 1400).

Once the control unit 812 has identified the relevant activation switch unit 806, 808, the control unit 812 generates or invokes one or more switch control signals which cause the connection switch unit 810 to be activated, and the identified activation switch unit 806, 808 to be activated (e.g. causes the switches 807, 809 of the identified activation switch unit 806, 808 to be in a closed position), subsequent the connection switch unit 810 (block 1410 of method 1400). For example, as described above, in some cases the control unit 812 may generate a single oscillating signal (e.g. a square wave) for a predetermined period which is provided to the connection switch unit 810 and the appropriate activation switch unit 806, 808 to cause them to be activated in sequence.

The token-based approach described above means that the latency can be verified end to end without reference to a more complicated clock synchronisation or link-specific latency detection method. It also controls the risk that a computer system between the external computing device and the electrosurgical connection unit might get stuck repeating the same stale activation state and renders such behaviour harmless.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein. In view of the foregoing description it will be evident to a person skilled in the art that various modifications may be made within the scope of the invention.

1. An electrosurgical connection unit for a surgical robot arm, the electrosurgical connection unit comprising:
an input port connectable to an electrosurgical generator;
an output port connectable to an electrosurgical instrument attached to the surgical robot arm;
one or more activation switch units wherein activation of an activation switch unit of the one or more activation switch units causes an activation signal to be output from the input port, the activation signal is configured to, when the input port is connected to the electrosurgical generator, cause the electrosurgical generator to generate a driving electrosurgical signal which is received on the input port;
a connection switch unit, wherein activation of the connection switch unit causes a portion of the input port to be electrically connected to the output port such that, when the input port is connected to the electrosurgical generator, the driving electrosurgical signal received on the input port is output on the output port; and
a control unit configured to, in response to receiving a control signal from an external computing device, cause the connection switch unit to be activated and subsequently cause an activation switch unit of the one or more activation switch units to be activated.

2. The electrosurgical connection unit of claim 1, wherein the control unit is configured to cause the connection switch unit to be activated and subsequently cause the activation switch unit of the one or more activation switch units to be activated by outputting a first switch control signal to control the connection switch unit and a second switch control signal to control the activation switch unit.

3. The electrosurgical connection unit of claim 2, wherein the first and second switch control signals are the same.

4. The electrosurgical connection unit of claim 2, further comprising a first circuit that is situated between the control unit and the connection switch unit and is configured to convert the first switch control signal to a signal to activate the connection switch unit, and a second circuit situated between the control unit and the activation switch unit and configured to convert the second switch control signal to a signal to activate the activation switch unit, wherein the first and second circuits are configured to, when the first and second control signals are the same, activate the connection switch unit prior to activating the activation switch unit.

5. The electrosurgical connection unit of claim 4, wherein the first and second switch control signals are oscillating signals and the first and second circuits are first and second conversions circuits configured to convert an oscillating signal to a control signal, wherein the first conversion circuit is configured differently from the second conversion circuit such that when the first and second switch control signals are the same, the connection switch unit is activated prior to the activation switch unit.

6. (canceled)

7. The electrosurgical connection unit of claim 4, wherein the first and second switch control signals are oscillating signals and the first and second circuits are first and second conversions circuits configured to convert an oscillating signal to a control signal, wherein each of the first and second conversion circuits comprises a low pass filter and a timing constant of the low pass filter of the first conversion circuit differs from a timing constant of the low pass filter of the second conversion circuit.

8. The electrosurgical connection unit of claim 7, wherein the low pass filter of each of the first and second conversion circuits comprises a capacitor and one or more resistors, and a resistance value of at least one of the one or more resistors of the low pass filter of the first conversion circuit differs from a resistance value of a corresponding resistor of the low pass filter of the second conversion circuit.

9. The electrosurgical connection unit of claim 4, wherein the control unit is configured to, in response to ceasing to receive the control signal, deactivate the activation switch unit and subsequently deactivate the connection switch unit.

10. The electrosurgical connection unit of claim 9, wherein the control unit is configured to deactivate the activation switch unit and subsequently deactivate the connection switch unit by ceasing to output the first and second switch control signals, wherein the first and second circuits are configured to, in response to ceasing to receive the first and second control signals at a same time, cause the connection switch unit to be deactivated subsequent to causing the activation switch unit to be deactivated.

11. (canceled)

12. The electrosurgical connection unit of claim 1, wherein:
the input port is coupled to an input active wire so as to receive the driving electrosurgical signal from the input port;
the output port is coupled to an output active wire so as to provide the driving electrosurgical signal to the output port; and
the connection switch unit, when activated, electrically connects the input active wire and the output active wire.

13. The electrosurgical connection unit of claim 1, wherein, when the output port is connected to the electrosurgical instrument outputting the driving electrosurgical signal on the output port causes a return electrosurgical signal to be received on the output port, and the electrosurgical connection unit further comprises a second connection switch unit, and activation of the second connection switch unit causes another portion of the input port to be electrically connected to the output port such that the return electrosurgical signal received on the output port is output on the input port.

14. The electrosurgical connection unit of claim 1, wherein the input port is coupled to (i) an input active wire in order to receive the driving electrosurgical signal from the input port and (ii) one or more control wires in order to transmit an activation signal to the input port, and when the activation switch unit is activated the input active wire is electrically connected to one of the one or more control wires to generate the activation signal.

15. The electrosurgical connection unit of claim 14, wherein each activation switch unit of the one or more activation switch units comprises one or more switches in series and one end of the one or more switches is coupled to the input active wire and the other end of the one or more switches is coupled to one of the one or more control wires.

16. The electrosurgical connection unit of claim 1, wherein the one or more activation switch units comprises a first activation switch unit and a second activation switch unit, wherein, when the input port is connected to the electrosurgical generator, activating the first activation switch unit causes a first activation signal to be transmitted to the electrosurgical generator which causes the electrosurgical generator to output a driving electrosurgical signal with a first waveform, and activating the second activation switch unit causes a second activation signal to be transmitted to the electrosurgical generator which causes the electrosurgical generator to output a driving electrosurgical signal with a second waveform.

17. (canceled)

18. The electrosurgical connection unit of claim 1, further comprising an isolation device to form an isolation barrier between (i) the one or more activation switch units and the connection switch unit and (ii) the control unit.

19. The electrosurgical connection unit of claim 1, further comprising an alternating current coupling circuit for each activation switch unit of the one or more activation switch units, each alternating current coupling circuit being situated between the control unit and the corresponding activation switch unit of the one or more activation switch units, each alternating current coupling circuit configured to receive an alternating current switch control signal output by the control unit and generate a direct current filtered version of the alternating current switch control signal.

20. The electrosurgical connection unit of claim 1, further comprising a measurement unit configured to measure a parameter of an activation switch unit of the one or more activation switch units or the connection switch unit and output measurement information to the control unit, the measurement information enabling a determination to be made as whether the activation switch unit or the connection switch unit is operating as expected.

21. The electrosurgical connection unit of claim 1, further comprising a capacitance emulation unit configured to, when the electrosurgical generator is connected to the input port, add or subtract capacitance to an electrical path between the electrosurgical connection unit and the electrosurgical generator.

22. The electrosurgical connection unit of claim 1, wherein the control unit is configured to periodically generate a token comprising information indicating a time at which the token was generated, and transmit the token to an external computing device, and the control signal is a modified version of one of the tokens, wherein the control unit is configured to only activate the connection switch unit and the activation switch unit of the one or more activation switch units in response to receiving the modified version of the token when, at the time the modified version of the token is received at the control unit, an elapsed time since the token was generated is less than a threshold.

23. (canceled)

24. A surgical robotic system comprising:
a surgical robot arm comprising the electrosurgical connection unit of claim 1;
the electrosurgical generator connected to the input port of the electrosurgical connection unit; and
the electrosurgical instrument connected to the output port of the electrosurgical connection unit.
